(19) United States
(12) Patent Application Publication
(10) Pub. No.: US 2020/0069818 A1
(43) Pub. Date: Mar. 5, 2020

(54) CRISPR/CAS9-BASED COMPOSITIONS AND METHODS FOR TREATING CANCER

(71) Applicant: THE JOHNSON HOPKINS UNIVERSITY, Baltimore, MD (US)

(72) Inventors: Vinod JASKULA-RANGA, Cambridge, MA (US); Donald ZACK, Baltimore, MD (US); Fred BUNZ, Baltimore, MD (US)

(73) Assignee: The Johns Hopkins University, Baltimore, MD (US)

(21) Appl. No.: 16/315,468

(22) PCT Filed: Jul. 5, 2017
(86) PCT No.: PCT/US2017/040696

§ 371 Date: Jan. 4, 2019

Related U.S. Application Data
(60) Provisional application No. 62/358,339, filed on Jul. 5, 2016.

(51) Int. Cl. A61K 48/00; A61P 35/00; C12N 15/113; C12N 15/10; C12N 15/86; C12N 9/16; A61K 35/761

(52) U.S. Cl. CPC A61K 48/0066; A61P 35/00; C12N 15/1135; A61K 45/06; C12N 15/86; C12N 9/16; A61K 35/761; C12N 15/102

(57) ABSTRACT

Described herein are methods for preventing, inhibiting, or treating cancer in a subject. Also provided herein are methods of altering expression of one or more gene products in a cell, such as a cancer cell. Such methods may comprise utilizing a modified nuclease system, such as Clustered Regularly Interspaced Short Palindromic Repeats (CRISPR)/CRISPR associated (Cas) 9 (CRISPR-Cas9) comprising a bidirectional HI promoter and gRNAs directed to oncogenes (rAAV-Onco-CRISPR) or tumor suppressor genes (rAAV-TSG) packaged in a compact adeno-associated virus (AAV) particle. Such methods may comprise co-administering or concurrently providing a recombinant adeno-associated virus-packaging adenovirus (Ad-rAAVpack) with the nuclease system.

### CROSS-REFERENCE

This application claims the benefit of U.S. Provisional Application No. 62/358,339, filed Jul. 5, 2016, the entirety of which is hereby incorporated by reference.

### STATEMENT OF GOVERNMENT SUPPORT

This invention was made with government support under R01CA157535 awarded by the National Cancer Institute. The government has certain rights in the invention.

### BACKGROUND

One of the major challenges of successful and effective targeting of a cancer-related or cancer-specific molecule (e.g. gene, protein, enzyme) is the lack of specificity. Current chemotherapeutics and agents under preclinical validation are effective in the inhibition of a chosen molecule but are not specific to the target. This, in fact, is the principal causal factor for the unwanted and undesirable toxicities experienced with chemotherapeutics in general. While the gene therapeutic strategies such as shRNA or siRNA are very specific to the molecular target, their selective delivery to the tumor is a major challenge.

Furthermore, the siRNA has the limitation of inactivating or neutralizing the target at 1:1 ratio which would necessitate a constant and high levels of delivery of specific RNA to the tumor. The shRNA on the other hand, once introduced into the tumor, could integrate into host genome and produce a continuous antisense oligos that can interfere with specific target.

Preclinical reports indicate that molecular targeting of cancer significantly improves therapeutic efficacy (Gharwan, H. & Groninger, H. Nat. Rev. Clin. Oncol. (2015).). Yet, successful clinical translation of majority of anticancer agents remains a challenge (Rothenberg, M L et al. Nat. Rev. Cancer. 3, 303-309 (2003); Le Tourneau, C et al. Target Oncol. 5, 65-72 (2010)). Although nucleic acid-based, antisense therapeutic approaches (e.g. siRNA, shRNA) enjoy superiority in molecular specificity and effective inhibition, certain inherent limitations hamper their success towards clinical application (Ganapathy-Kanniappan S et al. Mol Cancer. 2013; 12: 152, 4598-12-152., Pecot, C V et al. Nat. Rev. Cancer. 11, 59-67 (2011)).

Therefore, there is a strong need to develop innovative therapeutic strategies and compositions with enhanced target specificity in the treatment of cancer.

### SUMMARY

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{d} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{h}ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, available on the World Wide Web: http://www.ncbi.nlm.nih.gov/omim/ and in Online Mendelian Inheritance in Animals (OMIA), a database of genes, inherited disorders and traits in animal species (other than human and mouse), available on the World Wide Web: http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Described herein are methods for treating cancer. The methods use a modified nuclease system, such as Clustered Regularly Interspaced Short Palindromic Repeats (CRISPR)/CRISPR associated (Cas) 9 (CRISPR-Cas9), to therapeutically target oncogene mutations or to repair defective tumor suppressor genes. The CRISPR-Cas9-based gene editing can be used to inactivate or correct oncogene mutations causing cancer, thereby providing a gene therapy approach for treating the underlying causes of cancer.

Thus, one aspect of the invention relates to a method for preventing, inhibiting, or treating cancer in a subject, the method comprising administering to the subject a therapeutically effective amount of a nuclease system (e.g., CRISPR-Cas9) comprising a genome targeted nuclease (e.g., Cas9 protein) and a guide RNA comprising at least one targeted genomic sequence, such as an oncogenic mutation (e.g., rAAV-Onco-CRISPR) or tumor suppressor gene (e.g, rAAV-TSG). The method may further comprise co-administering an adenovirus with the capability to package recombinant adeno-associated viruses in vivo (e.g., adeno-associated virus-packaging adenovirus; “Ad-rAAVpack”) in conjuction or concurrently with rAAV-Onco-CRISPR or rAAV-TSG, as described herein.

Another aspect of the invention provides methods for preventing, inhibiting, or treating cancer which utilize a composition comprising a modification of a non-naturally occurring CRISPR-Cas system previously described in WO2015/195621 (herein incorporated by reference in its entirety). Such a modification uses certain gRNAs that target cancer oncogenic mutations, such as, but not limited, to KRAS, PIK3CA, or IDH1, or mutations in tumor suppressor genes. In some embodiments, the composition comprises (a) a non-naturally occurring nuclease system (e.g., CRISPR-Cas9) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, an adeno-associated virus-packaging adenovirus (e.g., Ad-rAAVpack) is concurrently or co-administered with the adeno-associated virus containing the nuclease system. Ad-rAAVpack could also be employed along with an rAAV that does not encode a nuclease-gRNA system, but instead encodes a gene that would promote destruction of the tumor or increased recognition of the tumor by the immune system. For example, Ad-rAAVPack could direct the packaging of a companion rAAV that encodes a transgene such as interferon-α or wild type p53. For example, the AAV-onco-CRISPR or AAV-TSG could be delievered alone or in tandem with Ad-rAAVPack. In contrast, Ad-rAAVPack can be used with any rAAV, whether or not it is engineered to deliver a nuclease-gRNA. In some embodiments, the nuclease system is packaged into a single adeno-associated virus (AAV) particle. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

Another aspect of the invention provides methods of altering expression of one or more gene products in a eukaryotic cell, wherein the cell comprises a DNA molecule encoding the one or more gene products, the method comprising introducing into the cell a modified non-naturally occurring CRISPR-Cas system previously described in WO2015/195621 (herein incorporated by reference in its entirety). Such a modification uses certain gRNAs that target oncogenic mutations, such as, but not limited, to KRAS, PIK3CA, or IDH1, or tumor suppressor genes. In some embodiments, the method comprising introducing into the cell a composition comprising (a) a non-naturally occurring nuclease system (e.g., CRISPR-Cas9) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, an adeno-associated virus-packaging adenovirus (e.g., Ad-rAAVpack) is concurrently or co-administered with the adeno-associated virus containing the nuclease system. In some embodiments, the nuclease system is packaged into a single adeno-associated virus (AAV) particle. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

One aspect of the invention relates to a method for preventing, inhibiting, or treating cancer in a subject in need thereof, the method comprising:

(a) providing a non-naturally occurring nuclease system comprising one or more vectors comprising: i) a promoter operably linked to at least one nucleotide sequence encoding a nuclease system guide RNA (gRNA), wherein the gRNA hybridizes with a target sequence of a DNA molecule in a cell of the subject, and wherein the DNA molecule encodes one or more oncogene products expressed in the cell; and ii) a regulatory element operable in a cell operably linked to a nucleotide sequence encoding a genome-targeted nuclease, wherein components (i) and (ii) are located on the same or different vectors of the system, wherein the gRNA targets and hybridizes with the target sequence and the nuclease cleaves the DNA molecule to alter expression of the one or more gene products; and (b) administering to the subject a therapeutically effective amount of the system.

In some embodiments, the method further comprises the step of providing a recombinant adeno-associated virus-packaging adenovirus (Ad-rAAVpack).

In some embodiments, the Ad-rAAVpack is provided concurrently or co-administered with the nuclease system.

In some embodiments, the system is CRISPR-Cas9.

In some embodiments, the system is packaged into a single adeno-associated virus (AAV) particle.

In some embodiments, the adeno-associated virus-packaging adenovirus comprises at least one deletion in an adenoviral gene.

In some embodiments, the adeno-associated virus-packaging adenovirus is selected from adenovirus serotype 2, adenovirus serotype 5, or adenovirus serotype 35.

In some embodiments, the packaging virus is adenovirus serotype 5.

In some embodiments, the adenoviral gene is selected from E1A, E1B, E2A, E2B, E3, E4, L1, L2, L3, L4, or L5.

In some embodiments, the adenoviral gene is E3.

In some embodiments, the system inactivates one or more gene products.

In some embodiments, the nuclease system excises at least one gene mutation.

In some embodiments, the promoter is a H1 promoter.

In some embodiments, the H1 promoter is bidirectional. The H1 promoter is both a pol II and pol III promoter.

In some embodiments, the H1 promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

In some embodiments, the genome-targeted nuclease is Cas9 protein.

In some embodiments, the Cas9 protein is codon optimized for expression in the cell.

In some embodiments, the promoter is operably linked to at least one, two, three, four, five, six, seven, eight, nine, or ten gRNA.

In some embodiments, the target sequence is an oncogene or tumor suppressor gene.

In some embodiments, the target sequence is an oncogene comprising at least one mutation.

In some embodiments, the target sequence is an oncogene selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110α, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, She, Nck, Src, Yes, Fyn, Wnt, Bcl2, PyV MT antigen, and SV40 T antigen.

In some embodiments, the target sequence is an oncogene selected from KRAS, PIK3CA, or IDH1.

In some embodiments, the target sequence is an oncogene, said oncogene is KRAS.

In some embodiments, the KRAS comprises a mutation selected from G13D, G12C, or G12D.

In some embodiments, the target sequence is selected from the group consisting of SEQ ID NO: 11-14, or combinations thereof.

In some embodiments, the target sequence is an oncogene, said oncogene is PIK3CA.

In some embodiments, the PIK3CA comprises a mutation selected from E345K, D549N, or H1047R.

In some embodiments, the target sequence is selected from the group consisting of SEQ ID NO: 15-18, or combinations thereof.

In some embodiments, the target sequence is an oncogene, said oncogene IDH1.

In some embodiments, the IDH1 comprises a R132H mutation.

In some embodiments, the gRNA sequence is selected from the group consisting of the nucleotide sequences set forth in SEQ ID NO: 1-10, or combinations thereof.

In some embodiments, the nuclease system is administered via systematic administration.

In some embodiments, the systematic administration is selected from the group consisting of oral, intravenous, intradermal, intraperitoneal, subcutaneous, and intramuscular administration.

In some embodiments, the nuclease system is administered intratumorally or peritumorally.

In some embodiments, the subject is treated with at least one additional anti-cancer agent.

In some embodiments, the anti-cancer agent is selected from the group consisting of paclitaxel, cisplatin, topotecan, gemcitabine, bleomycin, etoposide, carboplatin, docetaxel, doxorubicin, topotecan, cyclophosphamide, trabectedin, olaparib, tamoxifen, letrozole, and bevacizumab.

In some embodiments, the subject is treated with at least one additional anti-cancer therapy.

In some embodiments, the anti-cancer therapy is radiation therapy, chemotherapy, or surgery.

In some embodiments, the cancer is a solid tumor.

In some embodiments, the cancer is selected from the group consisting of brain cancer, gastrointestinal cancer, oral cancer, breast cancer, ovarian cancer, prostate cancer, pancreatic cancer, lung cancer, liver cancer, throat cancer, stomach cancer, and kidney cancer.

In some embodiments, the cancer is brain cancer.

In some embodiments, the subject is a mammal.

In some embodiments, the mammal is human.

In some embodiments, cell proliferation is inhibited or reduced in the subject.

In some embodiments, malignancy is inhibited or reduced in the subject.

In some embodiments, tumor necrosis is enhanced or increased in the subject.

Another aspect of the invention relates to a method of altering expression of one or more gene products in a cell, wherein the cell comprises a DNA molecule encoding the one or more gene products, the method comprising introducing into the cell: (i) a non-naturally occurring nuclease system comprising one or more vectors comprising: a) a promoter operably linked to at least one nucleotide sequence encoding a nuclease system guide RNA (gRNA), wherein the gRNA hybridizes with a target sequence of the DNA molecule; and

b) a regulatory element operable in the cell operably linked to a nucleotide sequence encoding a genome-targeted nuclease,

wherein components (a) and (b) are located on the same or different vectors of the system, wherein the gRNA targets and hybridizes with the target sequence and the nuclease cleaves the DNA molecule to alter expression of the one or more gene products.

In some embodiments, the method further comprises providing a recombinant adeno-associated virus-packaging adenovirus (Ad-rAAVpack).

In some embodiments, the Ad-rAAVpack is provided concurrently or co-administered with the nuclease system.

In some embodiments, the system is CRISPR-Cas9.

In some embodiments, the system is packaged into a single adeno-associated virus (AAV) particle.

In some embodiments, the packaging virus comprises at least one deletion in an adenoviral gene.

In some embodiments, the adeno-associated virus-packaging adenovirus is selected from adenovirus serotype 2, adenovirus serotype 5, or adenovirus serotype 35.

In some embodiments, the adenovirus packaging virus is adenovirus serotype 5.

In some embodiments, the adenoviral gene is selected from E1A, E1B, E2A, E2B, E3, E4, L1, L2, L3, L4, or L5.

In some embodiments, the adenoviral gene is E3.

In some embodiments, the system inactivates one or more gene products.

In some embodiments, the nuclease system excises at least one gene mutation.

In some embodiments, the promoter is a H1 promoter.

In some embodiments, the H1 promoter is bidirectional.

In some embodiments, the H1 promoter comprises: a) control elements that provide for transcription in one direction of at least one nucleotide sequence encoding a gRNA; and b) control elements that provide for transcription in the opposite direction of a nucleotide sequence encoding a genome-targeted nuclease.

In some embodiments, the genome-targeted nuclease is Cas9 protein.

In some embodiments, the Cas9 protein is codon optimized for expression in the cell.

In some embodiments, the promoter is operably linked to at least one, two, three, four, five, six, seven, eight, nine, or ten gRNA.

In some embodiments, the target sequence is an oncogene or tumor suppressor gene.

In some embodiments, the target sequence is a cancer driven gene selected from the group consisting of EP300, FBXW7, GATA1, GATA2, NOTCH1, NOTCH2, EXT1, EXT2, PTCH1, SMO, SPOP, SUFU, APC, AXIN1, CDH1, CTNNB1, EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1, SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1, PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ, GNAS, GPC3, KIT, MET, NKX21, PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3, FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC.

In some embodiments, the target sequence is an oncogene selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110α, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen.

In some embodiments, the target sequence is an oncogene selected from KRAS, PIK3CA, or IDH1.

In some embodiments, the target sequence is an oncogene, said oncogene is KRAS.

In some embodiments, the KRAS comprises a mutation selected from G13D, G12C, or G12D.

In some embodiments, the target sequence is selected from the group consisting of SEQ ID NO: 11-14, or combinations thereof.

In some embodiments, the target sequence is an oncogene, said oncogene is PIK3CA.

In some embodiments, the PIK3CA comprises a mutation selected from E345K, D549N, or H1047R.

In some embodiments, the target sequence is selected from the group consisting of SEQ ID NO: 15-18, or combinations thereof.

In some embodiments, the target sequence is an oncogene, said oncogene IDH1.

In some embodiments, the IDH1 comprises a R132H mutation.

In some embodiments, the gRNA sequence is selected from the group consisting of the nucleotide sequences set forth in SEQ ID NO: 1-10, or combinations thereof.

In some embodiments, the expression of the one or more gene products is decreased.

In some embodiments, the cell is a eukaryotic or non-eukaryotic cell.

In some embodiments, the eukaryotic cell is a mammalian or human cell.

In some embodiments, the eukaryotic cell is a cancerous cell.

In some embodiments, cell proliferation is inhibited or reduced in the cell.

In some embodiments, apoptosis is enhanced or increased in the cell.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows the relationship between Ad and AAV. Wild-type AAV can only propagate in Ad-infected cells. The compact, single-strand DNA genome of wild type AAV harbors two genes (right) flanked by inverted terminal repeats (ITR). The rest of the genetic elements required for AAV replication are provided in trans, by Ad. Wild type Ad causes self-limiting lytic infections, while modified viruses are frequently used as vectors for transgene delivery.

FIG. 2 shows a dual-virus gene delivery system. The recombinant virus Ad-rAAVpack expresses AAV rep and cap in addition to the other trans-factors required for AAV replication. Thus, Ad-rAAVpack facilitates the in vivo replication of co-infected rAAV. These companion rAAV can be armed with CRISPR-Cas9 elements or transgenes such as tumor suppressors. The two virus system can be used to propagate any type of rAAV in vivo.

FIG. 3 show a dual virus approach to oncolytic therapy. Ad-rAAVpack is applied to a tumor with a companion rAAV programmed to target a tumor-specific driver mutation. The rAAV will have no effect on tumors that do not harbor the mutation. Because of the host range restriction imposed by the E1B mutation, Ad-rAAVpack will selectively propagate in the cells of the tumor. Several mutations in E1B have been shown to confer this host range restriction. In some embodiments, a four amino acid mutation called sub19 is used (Chahal et al. (2013) 87:4432-44. Cells productively infected with Ad-rAAVpack will lyse, introducing new replicated Ad-rAAVpack and rAAV into the local environment. Cells infected with only the rAAV will be growth-inhibited because of the loss of the driver gene. Such cells may increase the immunogenicity of the tumor.

FIG. 4 contains three panels, A, B, and C, showing the use of RNA-directed nucleases for oncogenic inactivation for KRAS.

FIG. 5 contains two panels, A and B, showing the use of RNA-directed nucleases for oncogenic inactivation for PIK3CA.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Genome-editing technologies such as zinc fingers nucleases (ZFN) (Porteus, and Baltimore (2003) Science 300: 763; Miller et al. (2007) Nat. Biotechnol. 25:778-785; Sander et al. (2011) Nature Methods 8:67-69; Wood et al. (2011) Science 333:307) and transcription activator-like effectors nucleases (TALEN) (Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; Reyon et al. (2012) Nat. Biotechnol. 30:460-465) have empowered the ability to generate targeted genome modifications and offer the potential to correct disease mutations with precision. While effective, these technologies are encumbered by practical limitations as both ZFN and TALEN pairs require synthesizing large and unique recognition proteins for a given DNA target site. Several groups have recently reported high-efficiency genome editing through the use of an engineered type II CRISPR/Cas9 system that circumvents these key limitations (Cong et al. (2013) Science 339:819-823; Jinek et al. (2013) eLife 2:e00471; Mali et al. (2013) Science 339:823-826; Cho et al. (2013) Nat. Biotechnol. 31:230-232; Hwang et al. (2013) Nat. Biotechnol. 31:227-229). Unlike ZFNs and TALENs, which are relatively time consuming and arduous to make, the CRISPR constructs, which rely upon the nuclease activity of the Cas9 protein coupled with a synthetic guide RNA (gRNA), are simple and fast to synthesize and can be multiplexed. However, despite the relative ease of their synthesis, CRISPRs have technological restrictions related to their access to targetable genome space, which is a function of both the properties of Cas9 itself and the synthesis of its gRNA.

Cleavage by the CRISPR system requires complementary base pairing of the gRNA to a 20-nucleotide DNA sequence and the requisite protospacer-adjacent motif (PAM), a short nucleotide motif found 3′ to the target site (Jinek et al. (2012) Science 337: 816-821). One can, theoretically, target any unique N₂₀-PAM sequence in the genome using CRISPR technology. The DNA binding specificity of the PAM sequence, which varies depending upon the species of origin of the specific Cas9 employed, provides one constraint. Currently, the least restrictive and most commonly used Cas9 protein is from S. pyogenes, which recognizes the sequence NGG, and thus, any unique 21-nucleotide sequence in the genome followed by two guanosine nucleotides (N₂₀NGG) can be targeted. Expansion of the available targeting space imposed by the protein component is limited to the discovery and use of novel Cas9 proteins with altered PAM requirements (Cong et al. (2013) Science 339: 819-823; Hou et al. (2013) Proc. Natl. Acad. Sci. U.S.A., 110(39):15644-9), or pending the generation of novel Cas9 variants via mutagenesis or directed evolution. The second technological constraint of the CRISPR system arises from gRNA expression initiating at a 5′ guanosine nucleotide. Use of the type III class of RNA polymerase III promoters has been particularly amenable for gRNA expression because these short non-coding transcripts have well-defined ends, and all the necessary elements for transcription, with the exclusion of the 1+ nucleotide, are contained in the upstream promoter region. However, since the commonly used U6 promoter requires a guanosine nucleotide to initiate transcription, use of the U6 promoter has further constrained genomic targeting sites to GN19NGG (Mali et al. (2013) Science 339:823-826; Ding et al. (2013) Cell Stem Cell 12:393-394). Alternative approaches, such as in vitro transcription by T7, T3, or SP6 promoters, would also require initiating guanosine nucleotide(s) (Adhya et al. (1981) Proc. Natl. Acad. Sci. U.S.A. 78:147-151; Melton et al. (1984) Nucleic Acids Res. 12:7035-7056; Pleiss et al. (1998) RNA 4:1313-1317).

The presently disclosed subject matter relates to the modification of a CRISPR/Cas9 system to target an oncogenic mutation or tumor suppressor genes, which uses the H1 promoter to express guide-RNAs (gRNA or sgRNA) (WO2015/19561, herein incorporated by reference in its entirety). Such a modified CRISPR/Cas9 system in combination with a recombinant adeno-associated packaging virus can precisely target the oncogenic mutations in cancer, or facilitate the repair of a defective tumor suppressor gene, with greater efficacy, safety, and precision. Moreover, this modification provides a compact CRISPR/Cas9 system that allows for higher-resolution targeting of oncogenes over existing CRISPR, TALEN, or Zinc-finger technologies.

Thus, one aspect of the invention relates to a replication-competent adenovirus (Ad) that contains all of the trans-elements required for the replication and packaging of companion recombinant adeno-associated viruses (rAAV). This dual-virus system allows the replication of both viruses in tandem, and thereby facilitates the local propagation of rAAV at sites of in vivo administration. In some embodiments, the system comprises a mutation in the Ad E1B gene for partial restriction to cancer cells, and thus would facilitate the tumor-specific propagation of rAAV armed with driver gene-specific CRISPRs or other genetic elements designed to impede cancer cell proliferation.

The application of a dual Ad-AAV system for any use has not been reported. The novelty of this system is that therapeutic rAAV, which are uniformly non-replicating, can be made to be replication-competent.

Another aspect of the invention relates to compositions that may target gain of function mutations, which are known to contribute to the growth of many types of cancer. Many of the oncogenic mutations found in common cancers are recurrent in nature, that is, the exact same mutation occurs in a high proportion in cancers of a given type. Current efforts to target recurrent oncogene mutations commonly employ small molecule inhibitors, or strategies to achieve synthetic lethality to DNA damage. For example, the most prevalent oncogene, KRAS, has not been successfully targeted, and remains “undrugable”. Such compositions comprised gRNAs that direct efficient nuclease (i.e, Cas9)-mediated cleavage of several of the most commonly mutated sites. Notably, these compositions comprising gRNAs are highly specific for mutant alleles and would therefore have little effect on cells that harbor wild type alleles.

### I. Expression of CRISPR Guide RNAs Using the H1 Promoter

A. Compositions

In some embodiments, the presently disclosed methods for preventing, inhibiting, or treating cancer utilize a composition comprising a modification of a non-naturally occurring CRISPR-Cas system previously described in WO2015/195621 (herein incorporated by reference in its entirety). Such a modification uses certain gRNAs that target oncogenic mutations, such as, but not limited, to KRAS, PIK3CA, or IDH1, or tumor suppressor genes. In some embodiments, the composition comprises (a) a non-naturally occurring nuclease system (e.g., CRISPR-Cas9) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, an adeno-associated virus-packaging adenovirus (e.g., Ad-rAAVpack) is concurrently or co-administered with the adeno-associated virus containing the nuclease system (i.e., dual-virus packagaing system). In some embodiments, a single adeno-associated virus (AAV) particle will be employed without the packaging adenovirus. In some embodiments, the adeno-associated virus (AAV) may comprise any of the 11 human adeno-associated virus serotypes (e.g., serotypes 1-11). In some embodiments, the adenovirus (AAV) may comprise any of the 51 human adenovirus serotypes. In some embodiments, the adenovirus for in vivo packaging of rAAV (i.e., adeno-associated virus-packaging adenovirus) comprises at least one deletion in an adenoviral gene. In some embodiments, the packaging adenovirus is selected from adenovirus serotype 2, adenovirus serotype 5, or adenovirus serotype 35. In some embodiments, the adeno-associated packaging adenovirus is adenovirus serotype 5. In some embodiments, the adenoviral gene is selected from E1A, E1B, E2A, E2B, E3, E4, L1, L2, L3, L4, or L5. In some embodiments, the adenoviral gene is E3. In some embodiments, the system inactivates one or more gene products. In some embodiments, the nuclease system excises at least one gene mutation. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the Cas9 protein is codon optimized for expression in the cell. In some embodiments, the promoter is operably linked to at least one, two, three, four, five, six, seven, eight, nine, or ten gRNA. In some embodiments, the target sequence is an oncogene or tumor suppressor gene. In some embodiments, the target sequence is an oncogene comprising at least one mutation. In some embodiments, the target sequence is an oncogene selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110a, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen. In some embodiments, the target sequence is a cancer driver gene selected from the group consisting of EP300, FBXW7, GATA1, GATA2, NOTCH1, NOTCH2, EXT1, EXT2, PTCH1, SMO, SPOP, SUFU, APC, AXIN1, CDH1, CTNNB1, EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1, SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1, PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ, GNAS, GPC3, KIT, MET, NKX21, PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3, FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC. In some embodiments, the target sequence is an oncogene selected from KRAS, PIK3CA, or IDH1. In some embodiments, the target sequence is an oncogene, said oncogene is KRAS. In some embodiments, the KRAS comprises a mutation selected from G13D, G12C, or G12D. In some embodiments, the target sequence is selected from the group consisting of SEQ ID NO: 11-14, or combinations thereof. In some embodiments, the target sequence is an oncogene, said oncogene is PIK3CA. In some embodiments, the PIK3CA comprises a mutation selected from E345K, D549N, or H1047R. In some embodiments, the target sequence is selected from the group consisting of SEQ ID NO: 15-18, or combinations thereof. In some embodiments, the target sequence is an oncogene, said oncogene IDH1. In some embodiments, the IDH1 comprises a R132H mutation. In some embodiments, the gRNA sequence is selected from the group consisting of the nucleotide sequences set forth in SEQ ID NO: 1-10, or combinations thereof.

In some embodiments, the dual-virus packagaing system allows therapeutic rAAV to be iteratively replicated in vivo. In some embodiments, the dual-virus packagaing system comprises an Adenovirus 5 called Ad-rAAVpack, in which the rep and cap genes from wild type AAV replace the Ad E3 gene. Ad E3 normally functions to allow the virus to evade host immune responses, but is not required for lytic infection nor for packaging of AAV. Because the rep-cap cassette is only ˜1 kb larger than the E3 gene, the total size of Ad-rAAVpack is well within the published Ad packaging capacity. The Ad-rAAVpack has all of the trans-elements required for the replication and packaging of a companion rAAV (e.g., rAAV-TSG or rAAV-Onco-CRISPR). Co-infection of target tissues with Ad-rAAVpack and a therapeutic rAAV permits the rAAV to be propagated in vivo, potentially increasing the efficiency of transgene delivery.

In some embodiments, the presently disclosed preventing, inhibiting, or treating cancer utilizes a non-naturally occurring CRISPR-Cas system comprising one or more vectors comprising: a) an H1 promoter operably linked to at least one nucleotide sequence encoding a CRISPR-Cas system guide RNA (gRNA), wherein the gRNA hybridizes with a target sequence of a DNA molecule in a cell, and wherein the DNA molecule encodes one or more gene products expressed in the cell; and b) a regulatory element operable in a cell operably linked to a nucleotide sequence encoding a Cas9 protein, wherein components (a) and (b) are located on the same or different vectors of the system, wherein the gRNA targets and hybridizes with the target sequence and the Cas9 protein cleaves the DNA molecule to alter expression of the one or more gene products. In some embodiments, an adeno-associated virus-packaging adenovirus (e.g., Ad-rAAVpack) is concurrently or co-administered with the adeno-associated virus containing the CRISPR-Cas system.

In some embodiments, the presently disclosed subject matter provides a non-naturally occurring CRISPR-Cas system comprising one or more vectors comprising: a) an H1 promoter operably linked to at least one nucleotide sequence encoding a CRISPR-Cas system guide RNA (gRNA), wherein the gRNA hybridizes with a target sequence of a DNA molecule in a eukaryotic cell, and wherein the DNA molecule encodes one or more gene products expressed in the eukaryotic cell; and b) a regulatory element operable in a eukaryotic cell operably linked to a nucleotide sequence encoding a Type-II Cas9 protein, wherein components (a) and (b) are located on the same or different vectors of the system, whereby the gRNA targets and hybridizes with the target sequence and the Cas9 protein cleaves the DNA molecule, and whereby expression of the one or more gene products is altered. In some embodiments, an adeno-associated virus-packaging adenovirus (e.g., Ad-rAAVpack) is concurrently or co-administered with the adeno-associated virus containing the CRISPR-Cas system. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In another aspect, the Cas9 protein is codon optimized for expression in the eukaryotic cell. In a further aspect, the eukaryotic cell is a mammalian or human cell. In yet another aspect, the expression of the one or more gene products is decreased.

The presently disclosed subject matter also provides a non-naturally occurring CRISPR-Cas system comprising a vector comprising a bidirectional H1 promoter, wherein the bidirectional H1 promoter comprises: a) control elements that provide for transcription in one direction of at least one nucleotide sequence encoding a CRISPR-Cas system guide RNA (gRNA), wherein the gRNA hybridizes with a target sequence of a DNA molecule in a eukaryotic cell, and wherein the DNA molecule encodes one or more gene products expressed in the eukaryotic cell; and b) control elements that provide for transcription in the opposite direction of a nucleotide sequence encoding a Type-II Cas9 protein, whereby the gRNA targets and hybridizes with the target sequence and the Cas9 protein cleaves the DNA molecule, and whereby expression of the one or more gene products is altered. In some embodiments, an adeno-associated virus-packaging adenovirus (e.g., Ad-rAAVpack) is concurrently or co-administered with the adeno-associated virus containing the CRISPR-Cas system. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In another aspect, the Cas9 protein is codon optimized for expression in the eukaryotic cell. In a further aspect, the eukaryotic cell is a mammalian or human cell. In yet another aspect, the expression of the one or more gene products is decreased.

In some embodiments, the CRISPR complex comprises one or more nuclear localization sequences of sufficient strength to drive accumulation of the CRISPR complex in a detectable amount in the nucleus of a cell (e.g., eukaryotic cell). Without wishing to be bound by theory, it is believed that a nuclear localization sequence is not necessary for CRISPR complex activity in eukaryotes, but that including such sequences enhances activity of the system, especially as to targeting nucleic acid molecules in the nucleus. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes, or S. thermophilus Cas9, and may include mutated Cas9 derived from these organisms. The enzyme may be a Cas9 homolog or ortholog.

As used herein, “adenoviruses” are DNA viruses with a 36-kb genome. There are 51 human adenovirus serotypes that have been distinguished on the basis of their resistance to neutralization by antisera to other known adenovirus serotypes. Although the majority of adenoviral vectors are derived from serotypes 2 and 5, other serotypes such as type 35 may also be used. The wild type adenovirus genome is divided into early (E1 to E4) and late (L1 to L5) genes. Adenovirus vectors can be prepared to be either replication competent or non-replicating. Foreign genes can be inserted into three areas of the adenovirus genome (E1, E3, or E4) as well as behind the major late promoter. The ability of the adenovirus genome to direct production of adenoviruses is dependent on sequences in E1.

Examples of proteins involved in tumor suppression may include ATM (ataxia telangiectasia mutated), ATR (ataxia telangiectasia and Rad3 related), EGFR (epidermal growth factor receptor), ERBB2 (v-erb-b2 erythroblastic leukemia viral oncogene homolog 2), ERBB3 (v-erb-b2 erythroblastic leukemia viral oncogene homolog 3), ERBB4 (v-erb-b2 erythroblastic leukemia viral oncogene homolog 4), Notch 1, Notch2, Notch 3, or Notch 4, for example.

Examples of tumor suppressor genes that can be usefully editedt are Rb, P53, INK4a, PTEN, LATS, Apaf1, Caspase 8, APC, DPC4, KLF6, GSTPI, ELAC2/HPC2, NKX3.1, ATM, CHK2, ATR, BRCA1, BRCA2, MSH2, MSH6, PMS2, Ku70, Ku80, DNA/PK, XRCC4, Neurofibromatosis Type 1, Neurofibromatosis Type 2, Adenomatous Polyposis Coli, tWilms tumor-suppressor protein, Patched, STAG2, and FHIT.

Examples of recombinant oncogenes useful in the present invention include Her2, KRAS, HRAS, NRAS, EGFR, MDM2, TGF-β, RhoC, AKT, c-myc, β-catenin, PDGF, C-MET, PI3K-110α, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen. Preferred oncogenes are Her2, C-MET, PI3K-CA and AKT, and Her2 (also known as neu or ErbB2 (v-erb-b2 erythroblastic leukemia viral oncogene homolog 2)).

As used herein, “cancer driver genes” encompass the cancer genes including, but not limited to, EP300, FBXW7, GATA1, GATA2, NOTCH1, NOTCH2, EXT1, EXT2, PTCH1, SMO, SPOP, SUFU, APC, AXIN1, CDH1, CTNNB1, EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1. SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1, PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ, GNAS, GPC3, KIT, MET, NKX21, PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3, FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC. See also comprehensive list in Vogelstein et al. (2013) Science 339:1546

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell.

Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the presently disclosed subject matter in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed.

Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel (1990) Gene Expression Technology: Methods in Enzymology 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific.

In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (e.g., Boshart et al. (1985) Cell 41:521-530), the SV40 promoter, the dihydrofolate reductase promoter, the β-actin promoter, the phosphoglycerol kinase (PGK) promoter, and the EF1α promoter.

Also encompassed by the term “regulatory element” are enhancer elements, such as WPRE; CMV enhancers; the R-U5′ segment in LTR of HTLV-I (Takebe et al. (1988) Mol. Cell. Biol. 8:466-472); SV40 enhancer; and the intron sequence between exons 2 and 3 of rabbit β-globin (O'Hare et al. (1981) Proc. Natl. Acad. Sci. USA. 78(3):1527-31). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). Advantageous vectors include lentiviruses and adeno-associated viruses, and types of such vectors can also be selected for targeting particular types of cells.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

In aspects of the presently disclosed subject matter the terms “chimeric RNA”, “chimeric guide RNA”, “guide RNA”, “single guide RNA” and “synthetic guide RNA” are used interchangeably and refer to the polynucleotide sequence comprising the guide sequence. The term “guide sequence” refers to the about 20 bp sequence within the guide RNA that specifies the target site and may be used interchangeably with the terms “guide” or “spacer”.

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 900, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified, for example, disulfide bond formation, glycosylation, lipidation, acetylation, phosphorylation, or any other manipulation, such as conjugation with a labeling component.

As used herein the term “amino acid” includes natural and/or unnatural or synthetic amino acids, including glycine and both the D or L optical isomers, and amino acid analogs and peptidomimetics.

The practice of the present presently disclosed subject matter employs, unless otherwise indicated, conventional techniques of immunology, biochemistry, chemistry, molecular biology, microbiology, cell biology, genomics and recombinant DNA, which are within the skill of the art (Sambrook, Fritsch and Maniatis (1989) Molecular Cloning: A Laboratory Manual, 2nd edition; Ausubel et al., eds. (1987) Current Protocols in Molecular Biology); MacPherson et al., eds. (1995) Methods in Enzymology (Academic Press, Inc.): PCR 2: A Practical Approach); Harlow and Lane, eds. (1988) Antibodies, A Laboratory Manual; Freshney, ed. (1987) Animal Cell Culture).

Several aspects of the presently disclosed subject matter relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia co/i with vectors containing constitutive or inducible promoters directing the expression of either fusion or non-fusion proteins.

Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson (1988) Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A. respectively, to the target recombinant protein.

Examples of suitable inducible non-fusion E. coli expression vectors include pTrc (Amrann et al. (1988) Gene 69:301-315) and pET 11d (Studier et al. (1990) Gene Expression Technology: Methods in Enzymology 185, Academic Press, San Diego, Calif.).

In some embodiments, a vector is a yeast expression vector. Examples of vectors for expression in yeast Saccharomyces cerivisae include pYepSec1 (Baldari, et al. (1987) EMBO J. 6: 229-234), pMFa (Kuijan and Herskowitz (1982) Cell 30: 933-943), pJRY88 (Schultz et al. (1987) Gene 54: 113-123), pYES2 (Invitrogen Corporation, San Diego, Calif.), and picZ (InVitrogen Corp, San Diego, Calif.).

In some embodiments, a vector is capable of driving expression of one or more sequences in mammalian cells using a mammalian expression vector. Examples of mammalian expression vectors include pCDM8 (Seed (1987) Nature 329: 840) and pMT2PC (Kaufman et al. (1987) EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook et al. (1989) Molecular Cloning: A Laboratory Manual. 2nd ed., Cold Spring Harbor Laboratory, Cold Spring Harbor Laboratory Press, Cold Spring Harbor, N.Y.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1: 268-277), lymphoid-specific promoters (Calame and Eaton (1988) Adv. Immunol. 43: 235-275), in particular promoters of T cell receptors (Winoto and Baltimore (1989) EMBO J. 8: 729-733) and immunoglobulins (Baneiji et al. (1983) Cell 33: 729-740; Queen and Baltimore (1983) Cell 33: 741-748), neuron-specific promoters (e.g., the neurofilament promoter; Byrne and Ruddle (1989) Proc. Natl. Acad. Sci. USA 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249: 374-379) and the α-fetoprotein promoter (Campes and Tilghman (1989) Genes Dev. 3: 537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al. (1987) J. Bacteriol., 169:5429-5433; and Nakata et al. (1989) J. Bacteriol., 171:3553-3556), and associated genes. Similar interspersed SSRs have been identified in Haloferax mediterranei, Streptococcus pyogenes, Anabaena, and Mycobacterium tuberculosis (Groenen et al. (1993) Mol. Microbiol., 10:1057-1065; Hoe et al. (1999) Emerg. Infect. Dis., 5:254-263; Masepohl et al. (1996) Biochim. Biophys. Acta 1307:26-30; and Mojica et al. (1995) Mol. Microbiol., 17:85-93). The CRISPR loci typically differ from other SSRs by the structure of the repeats, which have been termed short regularly spaced repeats (SRSRs) (Janssen et al. (2002) OMICSJ. Inleg. Biol., 6:23-33; and Mojica et al. (2000) Afol. AMicrobiol., 36:244-246). In general, the repeats are short elements that occur in clusters that are regularly spaced by unique intervening sequences with a substantially constant length (Mojica et al. (2000) Mol. Microbhiol., 36:244-246). Although the repeat sequences are highly conserved between strains, the number of interspersed repeats and the sequences of the spacer regions typically differ from strain to strain (van Embden et al. (2000), J. Bacteriol., 182:2393-2401). CRISPR loci have been identified in more than 40 prokaryotes (e.g., Jansen et al. (2002) Mol. Microbiol., 43:1565-1575; and Mojica et al. (2005)J. Mol. Evol. 60:174-82) including, but not limited to Aeropyrm, Pyrobaculum, Sulfolobus, Archaeoglobus, Halocarcula, Methanohacteriumn, Methanococcus, Methanosarcina, Afethanopyrus, Pyrococcus, Picrophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, AMethylococcus, Pasteurella, Photobacterium, Salmonella, Xanthomonas, Yersinia, Ireponema, and Thermotoga.

In general, “CRISPR system” refers collectively to transcripts and other elements involved in the expression of or directing the activity of CRISPR-associated (“Cas”) genes, including sequences encoding a Cas gene, a guide sequence (also referred to as a “spacer” in the context of an endogenous CRISPR system), or other sequences and transcripts from a CRISPR locus. In some embodiments, one or more elements of a CRISPR system is derived from a type 1, type II, or type Ill CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the presently disclosed subject matter, an exogenous template polynucleotide may be referred to as an editing template. In an aspect of the presently disclosed subject matter the recombination is homologous recombination.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known, for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae.

In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura et al. (2000) Nucl. Acids Res. 28:292. Computer algorithms for codon optimizing a particular sequence for expression in a particular host cell are also available, such as Gene Forge (Aptagen, Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. A guide sequence may be selected to target any target sequence. In some embodiments, the target sequence is a sequence within a genome of a cell. Exemplary target sequences include those that are unique in the target genome. For example, in some embodiments, the target sequence is an oncogene (e.g., having an oncogenic mutationa) or tumor suppressor gene. In some embodiments, the target sequence is an oncogene comprising at least one mutation. In some embodiments, the target sequence is an oncogene selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110α, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen. In some embodiments, the target sequence is a cancer driver gene selected from the group consisting of EP300, FBXW7, GATA1, GATA2, NOTCH1, NOTCH2, EXT1, EXT2, PTCH1, SMO, SPOP, SUFU, APC, AXIN1, CDH1, CTNNB1, EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1, SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1, PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ, GNAS, GPC3, KIT, MET, NKX21, PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3, FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC. In some embodiments, the target sequence is an oncogene selected from KRAS, PIK3CA, or IDH1. In some embodiments, the target sequence is an oncogene, said oncogene is KRAS. In some embodiments, the KRAS comprises a mutation selected from G13D, G12C, or G12D. In some embodiments, the target sequence is selected from the group consisting of SEQ ID NO: 11-14, or combinations thereof. In some embodiments, the target sequence is an oncogene, said oncogene is PIK3CA. In some embodiments, the PIK3CA comprises a mutation selected from E345K, D549N, or H1047R. In some embodiments, the target sequence is selected from the group consisting of SEQ ID NO: 15-18, or combinations thereof. In some embodiments, the target sequence is an oncogene, said oncogene IDH1. In some embodiments, the IDH1 comprises a R132H mutation. In some embodiments, the gRNA sequence is selected from the group consisting of the nucleotide sequences set forth in SEQ ID NO: 1-10, or combinations thereof.

In some embodiments, the target sequence may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% homologous to the nucleotide sequences set forth in SEQ ID NO: 11-18.

The term “homologous” refers to the “% homology” and is used interchangeably herein with the term “% identity” herein, and relates to the level of nucleic acid sequence identity when aligned using a sequence alignment program.

For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to about, 80%, 81%, 82%, 83%, 84%, 85%, 86%, 87%, 88%, 89%, 90%, 91%, 92%, 93%, 94%, 95%, 96%, 97%, 98%, 99.1%, 99.2%, 99.3%, 99.4%, 99.5%, 99.6%, 99.7%, 99.8% or more sequence identity to the nucleotide sequences set forth in SEQ ID NO: 1-18.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CR ISPR enzyme are described in US20110059502, incorporated herein by reference. In some embodiments, a tagged CRISPR enzyme is used to identify the location of a target sequence.

In an aspect of the presently disclosed subject matter, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the presently disclosed subject matter, the DNA molecule encoding the gene product may be introduced into the cell via a vector. In a preferred embodiment of the presently disclosed subject matter the gene product is luciferase. In a further embodiment of the presently disclosed subject matter the expression of the gene product is decreased.

Generally, promoter embodiments of the present presently disclosed subject matter comprise: 1) a complete Pol III promoter, which includes a TATA box, a Proximal Sequence Element (PSE), and a Distal Sequence Element (DSE); and 2) a second basic Pol III promoter that includes a PSE and TATA box fused to the 5′ terminus of the DSE in reverse orientation. The TATA box, which is named for its nucleotide sequence, is a major determinant of Pol III specificity. It is usually located at a position between nt. −23 and −30 relative to the transcribed sequence, and is a primary determinant of the beginning of the transcribed sequence. The PSE is usually located between nt. −45 and −66. The DSE enhances the activity of the basic Pol III promoter. In the H1 promoter, there is no gap between the PSE and the DSE.

Bidirectional promoters consists of: 1) a complete, conventional, unidirectional Pol III promoter that contains 3 external control elements: a DSE, a PSE, and a TATA box; and 2) a second basic Pol III promoter that includes a PSE and a TATA box fused to the 5′ terminus of the DSE in reverse orientation. The TATA box, which is recognized by the TATA binding protein, is essential for recruiting Pol III to the promoter region. Binding of the TATA binding protein to the TATA box is stabilized by the interaction of SNAPc with the PSE. Together, these elements position Pol III correctly so that it can transcribe the expressed sequence. The DSE is also essential for full activity of the Pol III promoter (Murphy et al. (1992) Mol. Cell Biol. 12:3247-3261; Mittal et al. (1996) Mol. Cell Biol. 16:1955-1965; Ford and Hernandez (1997) J. Biol. Chem., 272:16048-16055; Ford et al. (1998) Genes, Dev., 12:3528-3540; Hovde et al. (2002) Genes Dev. 16:2772-2777). Transcription is enhanced up to 100-fold by interaction of the transcription factors Oct-1 and/or SBF/Staf with their motifs within the DSE (Kunkel and Hixon (1998) Nucl. Acid Res., 26:1536-1543). Since the forward and reverse oriented basic promoters direct transcription of sequences on opposing strands of the double-stranded DNA templates, the positive strand of the reverse oriented basic promoter is appended to the 5′ end of the negative strand of the DSE. Transcripts expressed under the control of the H1 promoter are terminated by an unbroken sequence of 4 or 5 T's.

In the H1 promoter, the DSE is adjacent to the PSE and the TATA box (Myslinski et al. (2001) Nucl. Acid Res. 29:2502-2509). To minimize sequence repetition, this promoter was rendered bidirectional by creating a hybrid promoter, in which transcription in the reverse direction is controlled by appending a PSE and TATA box derived from the U6 promoter. To facilitate construction of the bidirectional H1 promoter, a small spacer sequence may also inserted between the reverse oriented basic promoter and the DSE.

B. Methods

In some embodiments, the presently disclosed subject matter also provides a method of altering expression of one or more gene products in a eukaryotic cell, wherein the cell comprises a DNA molecule encoding the one or more gene products, the method comprising introducing into the cell a modified non-naturally occurring CRISPR-Cas system previously described in WO2015/195621 (herein incorporated by reference in its entirety). Such a modification uses certain gRNAs target oncogenic mutations, such as, but not limited, to KRAS, PIK3CA, or IDH1, or tumor suppressor genes. In some embodiments, the method comprising introducing into the cell a composition comprising (a) a non-naturally occurring nuclease system (e.g., CRISPR-Cas9) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell of the subject, and wherein the DNA molecule encodes one or more gene products expressed in the cell; and ii) a regulatory element operable in a cell operably linked to a nucleotide sequence encoding a genome-targeted nuclease (e.g., Cas9 protein), wherein components (i) and (ii) are located on the same or different vectors of the system, wherein the gRNA targets and hybridizes with the target sequence and the nuclease cleaves the DNA molecule to alter expression of the one or more gene products. In some embodiments, an adeno-associated virus-packaging adenovirus (e.g., Ad-rAAVpack) is concurrently or co-administered with the adeno-associated virus containing the nuclease system (i.e., dual-virus packagaing system). In some embodiments, a single adeno-associated virus (AAV) particle will be employed without the packaging adenovirus. In some embodiments, the adeno-associated virus (AAV) may comprise any of the 11 human adeno-associated virus serotypes (e.g., serotypes 1-11). In some embodiments, the adenovirus (AAV) may comprise any of the 51 human adenovirus serotypes. In some embodiments, the adeno-associated virus-packaging adenovirus comprises at least one deletion in an adenoviral gene. In some embodiments, the packaging adenovirus is selected from adenovirus serotype 2, adenovirus serotype 5, or adenovirus serotype 35. In some embodiments, the adeno-associated virus-packaging virus is adenovirus serotype 5. In some embodiments, the adenoviral gene is selected from E1A, E1B, E2A, E2B, E3, E4, L1, L2, L3, L4, or L5. In some embodiments, the system inactivates one or more gene products. In some embodiments, the nuclease system excises at least one gene mutation. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the Cas9 protein is codon optimized for expression in the cell. In some embodiments, the promoter is operably linked to at least one, two, three, four, five, six, seven, eight, nine, or ten gRNA. In some embodiments, the target sequence is an oncogene or tumor suppressor gene. In some embodiments, the target sequence is an oncogene comprising at least one mutation. In some embodiments, the target sequence is selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110α, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen. In some embodiments, the target sequence is a cancer driver gene selected from the group consisting of EP300, FBXW7, GATA1, GATA2, NOTCH1, NOTCH2, EXT1, EXT2, PTCH1, SMO, SPOP, SUFU, APC, AXIN1, CDH1, CTNNB1, EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1, SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1. PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ. GNAS, GPC3, KIT, MET, NKX21, PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3, FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC. In some embodiments, the target sequence is an oncogene selected from KRAS, PIK3CA, or IDH1. In some embodiments, the target sequence is an oncogene, said oncogene is KRAS. In some embodiments, the KRAS comprises a mutation selected from G13D, G12C, or G12D. In some embodiments, the target sequence is selected from the group consisting of SEQ ID NO: 11-14, or combinations thereof. In some embodiments, the target sequence is an oncogene, said oncogene is PIK3CA. In some embodiments, the PIK3CA comprises a mutation selected from E345K, D549N, or H1047R. In some embodiments, the target sequence is selected from the group consisting of SEQ ID NO: 15-18, or combinations thereof. In some embodiments, the target sequence is an oncogene, said oncogene IDH1. In some embodiments, the IDH1 comprises a R132H mutation. In some embodiments, the gRNA sequence is selected from the group consisting of the nucleotide sequences set forth in SEQ ID NO: 1-10, or combinations thereof.

In some embodiments, the presently disclosed subject matter also provides a method of altering expression of one or more gene products in a cell, wherein the cell comprises a DNA molecule encoding the one or more gene products, the method comprising introducing into the cell a non-naturally occurring CRISPR-Cas system comprising one or more vectors comprising: a) an H1 promoter operably linked to at least one nucleotide sequence encoding a CRISPR-Cas system guide RNA (gRNA), wherein the gRNA hybridizes with a target sequence of the DNA molecule; and b) a regulatory element operable in the cell operably linked to a nucleotide sequence encoding a Cas9 protein, wherein components (a) and (b) are located on the same or different vectors of the system, wherein the gRNA targets and hybridizes with the target sequence and the Cas9 protein cleaves the DNA molecule to alter expression of the one or more gene products.

In some embodiments, the presently disclosed subject matter also provides a method of altering expression of one or more gene products in a eukaryotic cell, wherein the cell comprises a DNA molecule encoding the one or more gene products, the method comprising introducing into the cell a non-naturally occurring CRISPR-Cas system comprising one or more vectors comprising: a) an H1 promoter operably linked to at least one nucleotide sequence encoding a CRISPR-Cas system guide RNA (gRNA), wherein the gRNA hybridizes with a target sequence of the DNA molecule; and b) a regulatory element operable in the eukaryotic cell operably linked to a nucleotide sequence encoding a Type-II Cas9 protein, wherein components (a) and (b) are located on the same or different vectors of the system, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In some embodiments, the target sequence comprises the nucleotide sequence AN₁₉NGG or GN₁₉NGG. In another aspect, the Cas9 protein is codon optimized for expression in the cell. In yet another aspect, the Cas9 protein is codon optimized for expression in the eukaryotic cell. In a further aspect, the eukaryotic cell is a mammalian or human cell. In another aspect, the expression of the one or more gene products is decreased.

The presently disclosed subject matter also provides a method of altering expression of one or more gene products in a eukaryotic cell, wherein the cell comprises a DNA molecule encoding the one or more gene products, the method comprising introducing into the cell a non-naturally occurring CRISPR-Cas system comprising a vector comprising a bidirectional H1 promoter, wherein the bidirectional H1 promoter comprises: a) control elements that provide for transcription in one direction of at least one nucleotide sequence encoding a CRISPR-Cas system guide RNA (gRNA), wherein the gRNA hybridizes with a target sequence of the DNA molecule; and b) control elements that provide for transcription in the opposite direction of a nucleotide sequence encoding a Type-II Cas9 protein, whereby the gRNA targets and hybridizes with the target sequence and the Cas9 protein cleaves the DNA molecule, and whereby expression of the one or more gene products is altered. In one aspect, the target sequence can be a target sequence that starts with any nucleotide, for example, N₂₀NGG. In some embodiments, the target sequence comprises the nucleotide sequence AN₁₉NGG. In some embodiments, the target sequence comprises the nucleotide sequence GN₁₉NGG. In some embodiments, the target sequence comprises the nucleotide sequence CN₁₉NGG. In some embodiments, the target sequence comprises the nucleotide sequence TN₁₉NGG. In another aspect, the target sequence comprises the nucleotide sequence AN₁₉NGG or GN₁₉NGG. In another aspect, the Cas9 protein is codon optimized for expression in the cell. In yet another aspect, the Cas9 protein is codon optimized for expression in the eukaryotic cell. In a further aspect, the eukaryotic cell is a mammalian or human cell. In another aspect, the expression of the one or more gene products is decreased.

In some aspects, the presently disclosed subject matter provides methods comprising delivering one or more polynucleotides, such as or one or more vectors as described herein, one or more transcripts thereof, and/or one or proteins transcribed therefrom, to a host cell. In some aspects, the presently disclosed subject matter further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson (1992) Science 256:808-813; Nabel and Feigner (1993) TIBTECH 11:211-217; Mitani and Caskey (1993) TIBTECH 11:162-166; Dillon (1993) TIBTECH 11:167-175; Miller (1992) Nature 357:455-460; Van Brunt (1998) Biolechnology 6(10): 1149-1154; Vigne (1995) Restorative Neurology and Neuroscience 8:35-36, Kremer and Perricaudet (1995) British Medical Bulletin 51(1):31-44; Haddada et al. (1995) Current Topics in Microbiology and Immunology. Doerfler and Bohm (eds); and Yu et al. (1994) Gene Therapy 1:13-26.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (e.g., Crystal (1995) Science 270:404-410; Blaese et al. (1995) Cancer Gene Ther. 2:291-297: Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (e.g., Buchscher et al. (1992) J. Virol. 66:2731-2739; Johann et al. (1992) J. Virol. 66:1635-1640; Sommnerfelt et al. (1990) J. Virol. 176:58-59; Wilson et al. (1989) J. Virol. 63:2374-2378; Miller et al. (1991) J. Virol. 65:2220-2224; PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (e.g., West et al. (1987) Virology 160:38-47; U.S. Pat. No. 4,797,368; WO 93/24641; Kotin (1994) Human Gene Therapy 5:793-801; Muzyczka (1994) J. Clin. Invest. 94:1351. Construction of recombinant AAV vectors are described in a number of publications, including U.S. Pat. No. 5,173,414; Tratschin et al. (1985) Mol. Cell. Biol. 5:3251-3260; Tratschin et al. (1984) Mol. Cell. Biol. 4:2072-2081; Hermonat and Muzyczka (1984) Proc. Natl. Acad. Sci. U.S.A. 81:6466-6470; and Samulski et al. (1989) J. Virol. 63:03822-3828.

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging, transgene expression, and integration into the host genome. Viral DNA is packaged in a cell line, which contains a helper plasmid encoding the other AAV genes, namely rep and cap, but lacking ITR sequences. The cell line may be infected with adenovirus as a helper; 293 cells and their derivatives contain adenovirus DNA and therefore do not require adenoviral infection for AAV packaging. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-L. COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-MeI 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal. In some embodiments, the transgenic animal is a mammal, such as a mouse, rat, or rabbit. In certain embodiments, the organism or subject is a plant. Methods for producing transgenic animals are known in the art, and generally begin with a method of cell transfection, such as described herein.

In one aspect, the presently disclosed subject matter provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may even be re-introduced into the non-human animal.

In one aspect, the presently disclosed subject matter provides for methods of modifying a target polynucleotide in a eukaryotic cell. In some embodiments, the method comprises allowing a CRISPR complex to bind to the target polynucleotide to effect cleavage of the target polynucleotide thereby modifying the target polynucleotide, wherein the CRISPR complex comprises a CRISPR enzyme complexed with a guide sequence hybridized to a target sequence within the target polynucleotide.

In one aspect, the presently disclosed subject matter provides a method of modifying expression of a polynucleotide in a eukaryotic cell. In some embodiments, the method comprises allowing a CRISPR complex to bind to the polynucleotide such that the binding results in increased or decreased expression of the polynucleotide; wherein the CRISPR complex comprises a CRISPR enzyme complexed with a guide sequence hybridized to a target sequence within the polynucleotide.

In one aspect, the presently disclosed subject matter provides methods for using one or more elements of a CRISPR system. The CRISPR complex of the presently disclosed subject matter provides an effective means for modifying a target polynucleotide. The CRISPR complex of the presently disclosed subject matter has a wide variety of utility including modifying (e.g., deleting, inserting, translocating, inactivating, activating) a target polynucleotide in a multiplicity of cell types. As such the CRISPR complex of the presently disclosed subject matter has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide.

The target polynucleotide of a CRISPR complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex.

The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

Embodiments of the presently disclosed subject matter also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (McIvor et al. (2010) RNA Biol. 7(5):551-8). The CRISPR-Cas system may be harnessed to correct these defects of genomic instability.

C. Formulations

In one aspect, the present invention provides pharmaceutically acceptable compositions which comprise the dual virus packing system (i.e., rAAV (e.g., rAAV-Onco-CRISPR or rAAV-TSG) and Ad-rAAVpack), formulated together with one or more pharmaceutically acceptable carriers (additives) and/or diluents. In another aspect the compositions can be administered as such or in admixtures with pharmaceutically acceptable carriers and can also be administered in conjunction with other anti-cancer therapies, such as chemotherapeutic agents, scavenger compounds, radiation therapy, biologic therapy, and the like. Conjunctive therapy thus includes sequential, simultaneous and separate, or co-administration of the composition, wherein the therapeutic effects of the first administered has not entirely disappeared when the subsequent compound is administered.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

As set out above, certain embodiments of the compositions comprising the dual virus packaging system (i.e., rAAV (e.g., rAAV-Onco-CRISPR or rAAV-TSG) and Ad-rAAVpack)) may contain a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compositions comprising the dual virus packing system (i.e., rAAV (e.g., rAAV-Onco-CRISPR or rAAV-TSG) and Ad-rAAVpack) of the present invention may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The compositions comprising the dual virus packing system (i.e., rAAV (e.g., rAAV-Onco-CRISPR or rAAV-TSG) and Ad-rAAVpack) formulations include those suitable for intratumoral, oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, a formulation of compositions comprising the dual virus packing system (i.e., rAAV (e.g., rAAV-Onco-CRISPR or rAAV-TSG) and Ad-rAAVpack) can comprise other carriers to allow more stability, to allow more stability, different releasing properties in vivo, targeting to a specific site, or any other desired characteristic that will allow more effective delivery of the dual virus packing system (i.e., rAAV (e.g., rAAV-Onco-CRISPR or rAAV-TSG) and Ad-rAAVpack) to a subject or a target in a subject, such as, without limitation, liposomes, microspheres, nanospheres, nanoparticles, bubbles, micelle forming agents, e.g., bile acids, and polymeric carriers, e.g., polyesters and polyanhydrides. In certain embodiments, an aforementioned formulation renders orally bioavailable a compound of the present invention.

Liquid dosage formulations of the dual virus packing system (i.e., rAAV (e.g., rAAV-Onco-CRISPR or rAAV-TSG) and Ad-rAAVpack) include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an active ingredient. A compositions comprising the dual virus packing system (i.e., rAAV (e.g., rAAV-Onco-CRISPR or rAAV-TSG) and Ad-rAAVpack) of the present invention may also be administered as a bolus, electuary or paste.

In solid dosage forms (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Dosage forms for the topical or transdermal administration of compositions comprising the dual virus packing system (i.e., rAAV (e.g., rAAV-Onco-CRISPR or rAAV-TSG) and Ad-rAAVpack) of the present invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions suitable for parenteral or intratumoral administration can comprise sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, the above-described pharmaceutical compositions can be combined with other pharmacologically active compounds (“second active agents”) known in the art according to the methods and compositions provided herein. Second active agents can be large molecules (e.g., proteins) or small molecules (e.g., synthetic inorganic, organometallic, or organic molecules). In one embodiment, second active agents independently or synergistically help to treat cancer.

For example, chemotherapeutic agents are anti-cancer agents. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used: interleukins 1 through 18, including mutants and analogues; interferons or cytokines, such as interferons α, β, and γ; hormones, such as luteinizing hormone releasing hormone (LHRH) and analogues and, gonadotropin releasing hormone (GnRH); growth factors, such as transforming growth factor-β (TGF-β), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-α & β (TNF-α & β); invasion inhibiting factor-2 (IIF-2); bone morphogenetic proteins 1-7 (BMP 1-7); somatostatin; thymosin-α-1; γ-globulin; superoxide dismutase (SOD); complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegal1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In another embodiment, the composition of the invention may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

### II. Methods for Treating Cancer

The presently disclosed subject matter provides methods for preventing, inhibiting, or treating cancer in a subject (e.g., human) in need thereof. The method comprises the steps of: (a) providing a non-naturally occurring nuclease system (e.g., CRISPR-Cas9) comprising one or more vectors comprising: i) a promoter (e.g., bidirectional H1 promoter) operably linked to at least one nucleotide sequence encoding a nuclease system guide RNA (gRNA), wherein the gRNA hybridizes with a target sequence of a DNA molecule in a cell (e.g., cancer cell) of the subject, and wherein the DNA molecule encodes one or more gene products expressed in the cell; and ii) a regulatory element operable in a cell operably linked to a nucleotide sequence encoding a genome-targeted nuclease (e.g., Cas9), wherein components (i) and (ii) are located on the same or different vectors of the system, wherein the gRNA targets and hybridizes with the target sequence and the nuclease cleaves the DNA molecule to alter expression or inactivates of the one or more gene products; and (b) administering to the subject a therapeutically effective amount of the system. In some embodiments, an adeno-associated virus-packaging adenovirus (e.g., Ad-rAAVpack) is concurrently or co-administered with the adeno-associated virus containing the nuclease system (i.e., dual-virus packagaing system). In some embodiments, the system is packaged into a single adeno-associated virus (AAV) particle will be employed without the packaging adenovirus. In some embodiments, the adeno-associated virus (AAV) may comprise any of the 11 human adenovirus serotypes (e.g., serotypes 1-11). In some embodiments, the adeno-associated packaging adenovirus comprises at least one deletion in an adenoviral gene. In some embodiments, the packaging adenovirus is selected from adenovirus serotype 2, adenovirus serotype 5, or adenovirus serotype 35. In some embodiments, the packaging virus is adenovirus serotype 5. In some embodiments, the adenoviral gene is selected from E1A, E1B, E2A, E2B, E3, E4, L1, L2, L3, L4, or L5. In some embodiments, the adenoviral gene is E3. In some embodiments, the system inactivates one or more gene products. In some embodiments, the nuclease system excises at least one gene mutation. In some embodiments, the promoter comprises: a) control elements that provide for transcription in one direction of at least one nucleotide sequence encoding a gRNA; and b) control elements that provide for transcription in the opposite direction of a nucleotide sequence encoding a genome-targeted nuclease. In some embodiments, the Cas9 protein is codon optimized for expression in the cell. In some embodiments, the promoter is operably linked to at least one, two, three, four, five, six, seven, eight, nine, or ten gRNA. In some embodiments, the target sequence is an oncogene or tumor suppressor gene. In some embodiments, the target sequence is an oncogene comprising at least one mutation. In some embodiments, the target sequence is an oncogene selected from the group consisting of Her2, PIK3CA, KRAS, HRAS, IDH1, NRAS, EGFR, MDM2, TGF-β, RhoC, AKT, c-myc, β-catenin, PDGF, C-MET, PI3K-110a, CDK4, cyclin B1, cyclin D1, estrogen receptor gene, progesterone receptor gene, ErbB1 (v-erb-b2 erythroblastic leukemia viral oncogene homolog 1), ErbB3 (v-erb-b2 erythroblastic leukemia viral oncogene homolog 3), PLK3, KIRREL, ErbB4 (v-erb-b2 erythroblastic leukemia viral oncogene homolog 4), TGFα, ras-GAP, Shc, Nck, Src, Yes, Fyn, Wnt, Bcl2, PyV MT antigen, and SV40 T antigen. In some embodiments, the target sequence is a cancer driver gene selected from the group consisting of EP300, FBXW7, GATA1, GATA2, NOTCH1, NOTCH2, EXT1, EXT2, PTCH1, SMO, SPOP, SUFU. APC, AXIN1, CDH1, CTNNB1. EP300, FAM123B, GNAS, HNF1A, NF2, PRKAR1A, RNF43, SOX9, ARID1A, ARID1B, ARID2, ASXL1, ATRX, CREBBP, DNMT1, DNMT3A, EP300, EZH2, H3F3A, HIST1H3B, IDH1, IDH2, KDM5C, KDM6A, MEN1, MLL2, MLL3, NCOA3, NCOR1, PAX5, PBRM1, SETD2, SETBP1, SKP2, SMARCA4, SMARCB1, SPOP, TET2, WT1, AR, BCOR, CREBBP, DAXX, DICER1, GATA3, IKZF1, KLF4, LMO1, PHOX2B, PHF6, PRDM1, RUNX1, SBDS, SF3B1, SRSF2, U2AF1, ABL1, BCL2, CARD11, CASP8, CCND1, CDC73, CDK4, CDKN2A, CDKN2C, CYLD, DAXX, FUBP1, MDM2, MDM4, MED12, MYC, MYCL1, MYCN, MYD88, NFE2L2, NPM1, PPM1D, PPP2R1A, RB1, TNFAIP3, TRAF7, TP53, ALK, B2M, BRAF, CBL, CEBPA, CSF1R, CIC, EGFR, ERBB2, FGFR2, FGFR3, FH, FLT3, GNA11, GNAQ, GNAS, HRAS, KIT, KRAS, MAP2K1, MAP3K1, MET, NRAS, NF1, PDGFRA, PTPN11, RET, SDH5, SDH8, SDHC, SDHD, VHL, AKT1, ALK, B2M, CBL, CEBPA, CSF1R, EGFR, ERBB2, FGFR2, FGFR3, FH, FLCN, FLT3, GNA11, GNAQ, GNAS, GPC3, KIT, MET, NKX21. PRKAR1A, PIK3CA, PIK3R1, PDGFRA, PTEN, RET, SDH5, SDH8, SDHC, SDHD, STK11, TSC1, TSC2, TSHR, VHL, WAS, CRLF2, FGFR2, FGFR3. FLT3, JAK1, JAK2, JAK3, KIT, MPL, SOCS1, VHL, B2M, CEBPA, ERK1, GNA11, GNAQ, MAP2K4, MAP3K1, NKX21, TNFAIP3, TSHR, WAS, ACVR1B, BMPR1A, FOXL2, GATA1, GATA2, GNAS, EP300, MED12, SMAD2, SMAD4, ATM, BAP1, BLM, BRCA1, BRCA2, BRIP1, BUB1B, CHEK2, ERCC2, ERCC3, ERCC4, ERCC5, FANCA, FANCC, FANCD2, FANCE, FANCF, FANCG, MLH1, MSH2, MSH6, MUTYH, NBS1, PALB2, PMS1, PMS2, RECQL4, STAG2, TP53, WRN, XPA, and XPC. In some embodiments, the target sequence is an oncogene selected from KRAS, PIK3CA, or IDH1. In some embodiments, the target sequence is an oncogene, said oncogene is KRAS. In some embodiments, the KRAS comprises a mutation selected from G13D, G12C, or G12D. In some embodiments, the target sequence is selected from the group consisting of SEQ ID NO: 11-14, or combinations thereof. In some embodiments, the target sequence is an oncogene, said oncogene is PIK3CA. In some embodiments, the PIK3CA comprises a mutation selected from E345K, D549N, or H1047R. In some embodiments, the target sequence is selected from the group consisting of SEQ ID NO: 15-18, or combinations thereof. In some embodiments, the target sequence is an oncogene, said oncogene IDH1. In some embodiments, the IDH1 comprises a R132H mutation. In some embodiments, the gRNA sequence is selected from the group consisting of the nucleotide sequences set forth in SEQ ID NO: 1-10, or combinations thereof. In some embodiments, the nuclease system is administered via systematic administration. In some embodiments, the systematic administration is selected from the group consisting of oral, intravenous, intradermal, intraperitoneal, subcutaneous, and intramuscular administration. In some embodiments, the nuclease system is administered intratumorally or peritumorally. In some embodiments, the method of claim 1, wherein the subject is treated with at least one additional anti-cancer agent. In some embodiments, the anti-cancer agent is selected from the group consisting of paclitaxel, cisplatin, topotecan, gemcitabine, bleomycin, etoposide, carboplatin, docetaxel, doxorubicin, topotecan, cyclophosphamide, trabectedin, olaparib, tamoxifen, letrozole, and bevacizumab. In some embodiments, the subject is treated with at least one additional anti-cancer therapy. In some embodiments, the anti-cancer therapy is radiation therapy, chemotherapy, or surgery. In some embodiments, the cancer is a solid tumor. In some embodiments, the cancer is selected from the group consisting of brain cancer, gastrointestinal cancer, oral cancer, breast cancer, ovarian cancer, prostate cancer, pancreatic cancer, lung cancer, liver cancer, throat cancer, stomach cancer, and kidney cancer. In some embodiments, the cancer is brain cancer. In some embodiments, the subject is a mammal. In some embodiments, the mammal is human. In some embodiments, cell proliferation is inhibited or reduced in the subject. In some embodiments, malignancy is inhibited or reduced in the subject. In some embodiments, tumor necrosis is enhanced or increased in the subject.

The ability of E1B-deficient adenoviruses to productively infect and lyse tumor cells has been published. While the mechanisms that underlie this cancer cell tropism have proven to be more complicated than first thought, the oncolytic adenovirus developed by Onyx (known as Onyx-015) performed well in some clinical trials, and is currently marketed in China as Oncorine. In contrast with ONYX-015, Ad-rAAVpack is designed with a subtle E1B mutation that prevents the virus from suppressing the innate immune response to infection, but retains the ability to direct the export of viral RNA to the cytoplasm. This response, mediated by interferons, is typically lost in many cancer cells.

The application of Ad-rAAVpack to cancer presents several strategic options. The companion rAAV could contain a compact tumor suppressor, or an immune-stimulant like interferon. Alternatively the companion rAAV could be armed with a CRISPR-Cas9 system (e.g. AAV-H1-CRISPR system). The gRNAs included in such an rAAV could be programmed to specifically target an oncogenic mutation, or to facilitate the repair of a defective tumor suppressor gene.

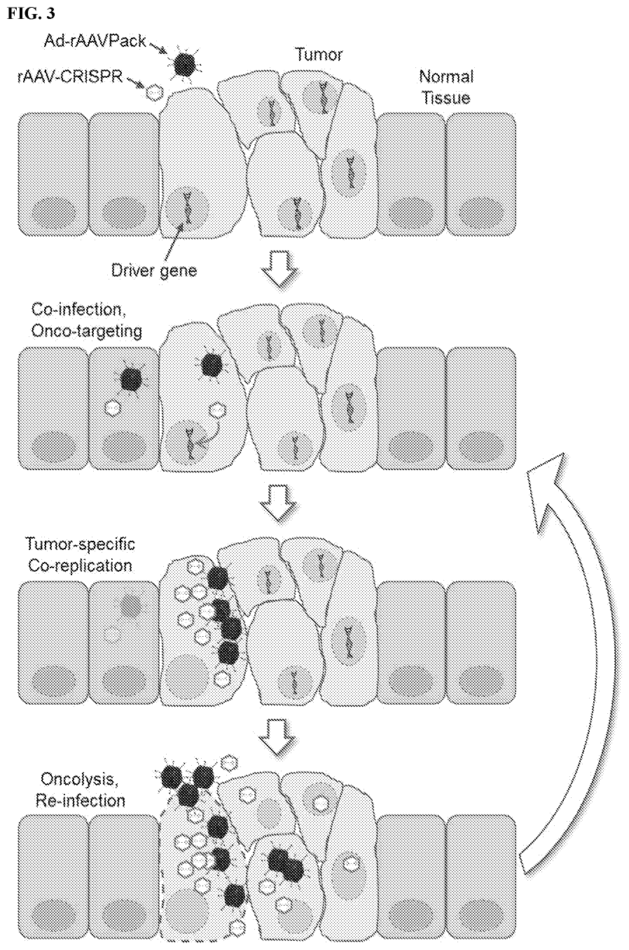

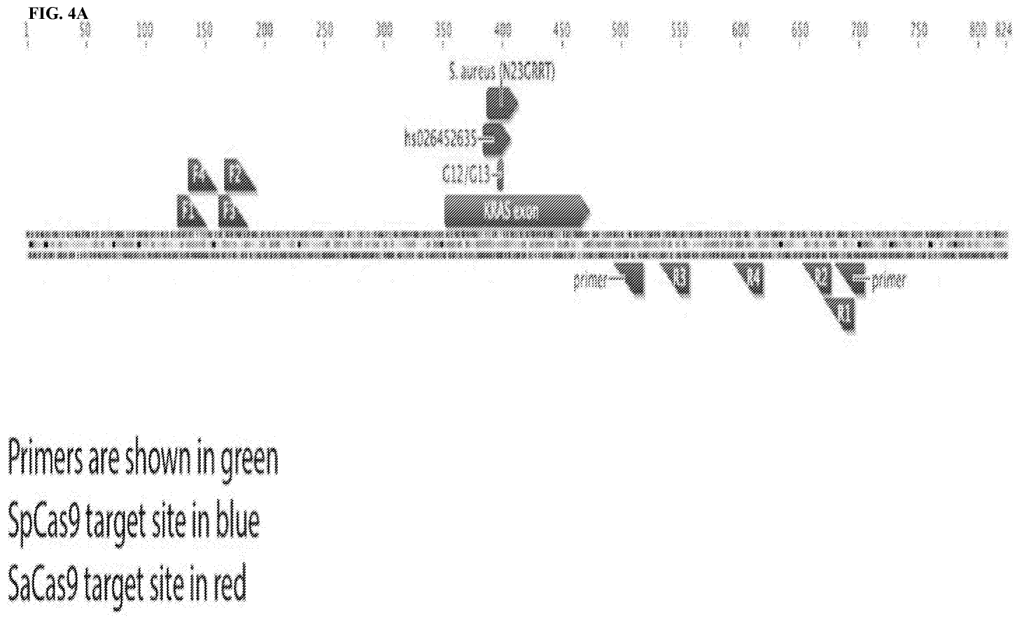

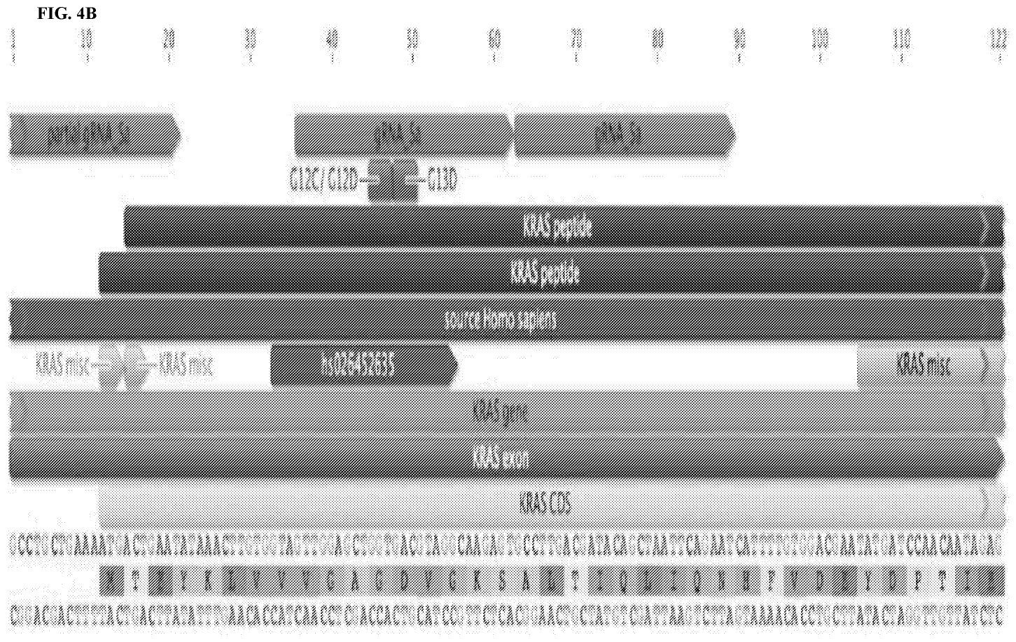

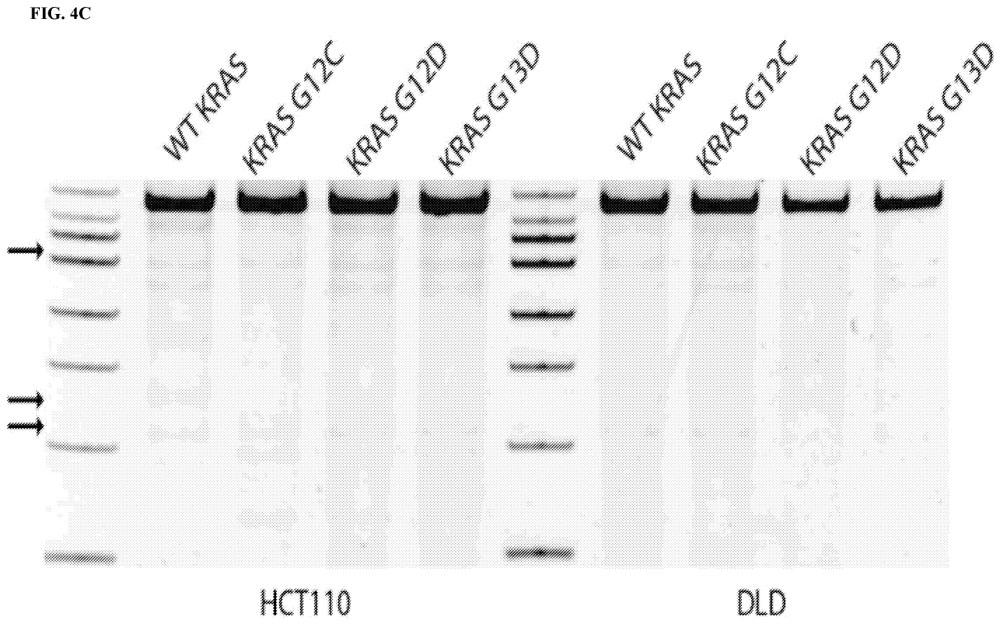

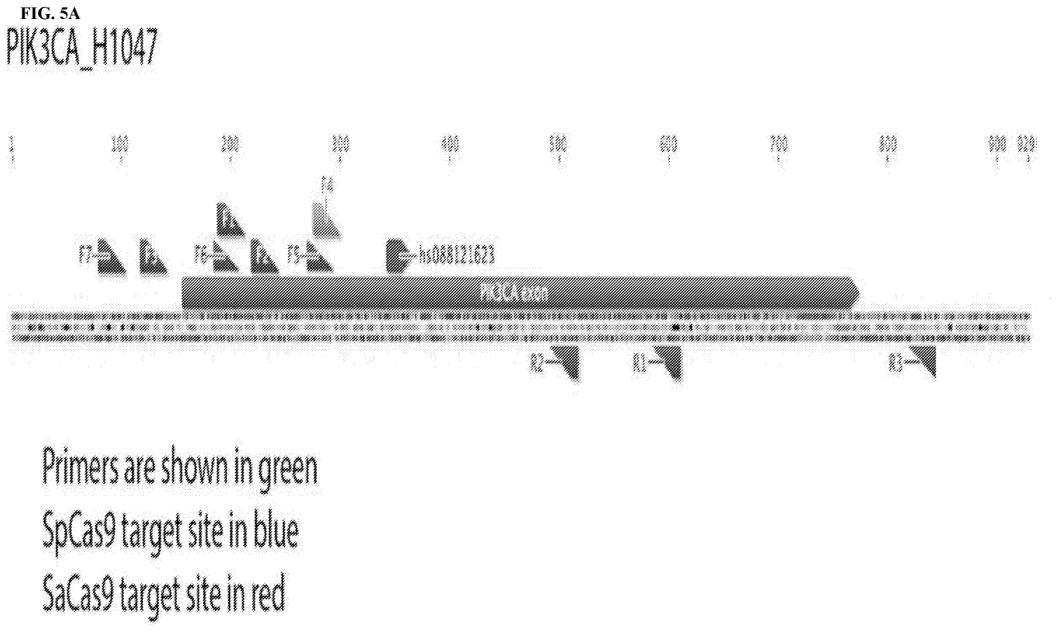

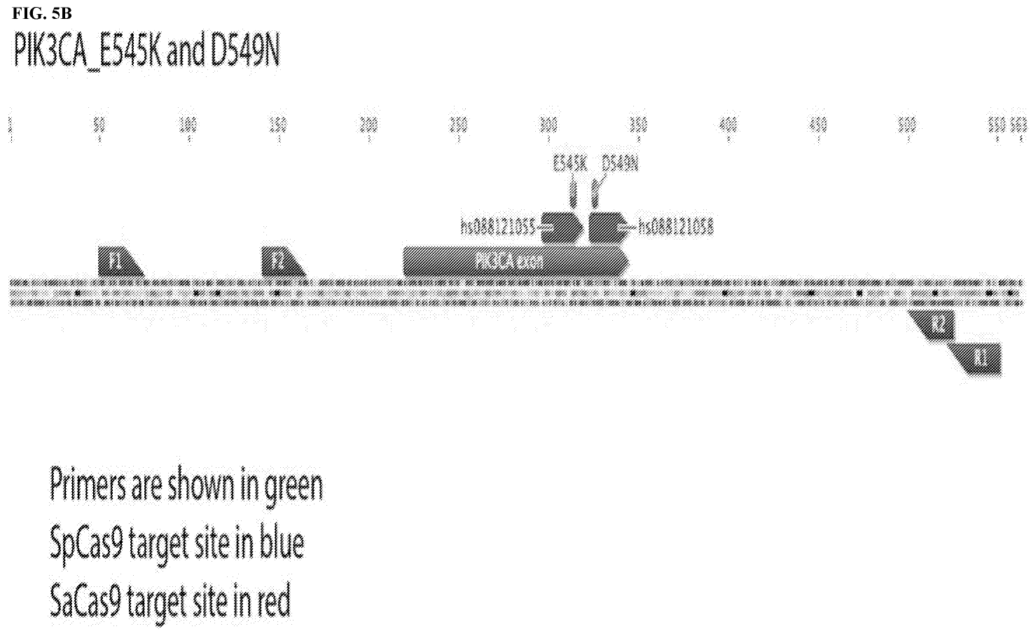

The predicted advantage of the dual virus system for cancer therapy over rAAV alone is the extent and duration of targeted gene delivery/targeted genetic alteration. A one-dose administration of therapeutic rAAV could probably target many cells in an accessible tumor. In the event that the proportion of cells thus modified is not sufficient to significantly affect the course of the disease, Ad-rAAVpack may be combined with rAAV. By matching the replicative potential of the tumor itself, the dual virus packagaing system may provide a unique opportunity to target a larger proportion of cancer cells, over a longer time scale. Without wishing to be bound by theory, an example of how this dual-virus oncolytic therapy might work is shown in FIG. 3.

To target oncogenic mutations, companion rAAV could be designed to inactivate recurrent oncogenic mutations in a highly specific fashion. Provided herein are a panel of gRNAs that may selectively disrupt cancer-associated oncogene forms of KRAS, PIK3CA and IDH1. Collectively, these specific mutations in KRAS and PIK3CA are found in the majority of cancers in the lung and throughout the GI tract. The IDH1 R132 mutation is found in about 30% of gliomas. These brain tumors are particularly refractory to all conventional forms of therapy. Each of these gRNA primarily targets the mutant allele without causing off-target effects in the remaining wild type allele.

The term “administering” means providing a pharmaceutical agent or composition to a subject, and includes, but is not limited to, administering by a medical professional and self-administering.

As used herein, the term “disorder” in general refers to any condition that would benefit from treatment with a compound against one of the identified targets, or pathways, including any disease, disorder, or condition that can be treated by an effective amount of a compound against one of the identified targets, or pathways, or a pharmaceutically acceptable salt thereof.

The term “cancer” as used herein refers to an abnormal growth of cells which tend to proliferate in an uncontrolled way and, in some cases, to metastasize (spread). The types of cancer include, but is not limited to, solid tumors (such as those of the bladder, bowel, brain, breast, endometrium, heart, kidney, lung, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

Additional non-limiting examples of cancers include, hepatocellular carcinoma (HCC), acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, lung cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, Wilms tumor.

As used herein, the term “treating” can include reversing, alleviating, inhibiting the progression of, preventing or reducing the likelihood of the disease, disorder, or condition to which such term applies, or one or more symptoms or manifestations of such disease, disorder or condition (e.g., cancer). In some embodiments, the treatment reduces cancer cells. For example, the treatment can reduce the cancer cells by at least 5%, 10%, 15%, 20%, 25%, 30%, 33%, 35%, 40%, 45%, 50%, 55%, 60%, 66%, 70%, 75%, 80%, 85%, 90%, 91%, 92%, 93%, 94%, 95%, 96%, 97%, 98%, 99% or more as compared to the cancer cells in a subject before undergoing treatment or in a subject who does not undergo treatment. In some embodiments, the treatment completely inhibits cancer cells in the subject.

In some embodiments, the system is packaged into a single adeno-associated virus (AAV) particle before administering to the subject. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The term “inhibit” or “inhibits” means to decrease, suppress, attenuate, diminish, arrest, or stabilize the development or progression of a disease, disorder, or condition, the activity of a biological pathway, or a biological activity, such as the growth of a solid malignancy, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the target, such as a growth of a solid malignancy, in a subject before the subject is treated. By the term “decrease” is meant to inhibit, suppress, attenuate, diminish, arrest, or stabilize a symptom of a cancer disease, disorder, or condition. It will be appreciated that, although not precluded, treating a disease, disorder or condition does not require that the disease, disorder, condition or symptoms associated therewith be completely eliminated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically-acceptable salts” refers to the relatively non-toxic, inorganic and organic acid addition salts of compounds.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The terms “subject” and “patient” are used interchangeably herein. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease.

The term “subject in need thereof” means a subject identified as in need of a therapy or treatment.

The terms “systemic administration,” “administered systemically,” “peripheral administration,” and “administered peripherally” mean the administration of a compound, drug or other material other than directly into the central nervous system, such that it enters the patient's system and, thus, is subject to metabolism and other like processes, for example, subcutaneous administration.

The term “therapeutic agent” or “pharmaceutical agent” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents is known in the art and may be identified by their effects. Certain therapeutic agents are capable of regulating cell proliferation and differentiation. Examples include chemotherapeutic nucleotides, drugs, hormones, non-specific (e.g. non-antibody) proteins, oligonucleotides (e.g., antisense oligonucleotides that bind to a target nucleic acid sequence (e.g., mRNA sequence)), peptides, and peptidomimetics.

The term “therapeutic effect” refers to a local or systemic effect in animals, particularly mammals, and more particularly humans, caused by a pharmacologically active substance.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The terms “tumor,” “solid malignancy,” or “neoplasm” refer to a lesion that is formed by an abnormal or unregulated growth of cells. Preferably, the tumor is malignant, such as that formed by a cancer.

The compositions, kits and detection, diagnosing and prognosing methods described above can be used to assist in selecting appropriate treatment regimen and to identify individuals that would benefit from more aggressive therapy.

As noted above, approaches to the treating cancers include surgery, immunotherapy, chemotherapy, radiation therapy, a combination of chemotherapy and radiation therapy, or biological therapy. Chemotherapeutics that have been used in the treatment of carcinomas include, but are not limited to, doxorubicin (Adriamycin), cisplatin, ifosfamide, and corticosteroids (prednisone). Often, these agents are given in combination to increase their effectiveness. Combinations used to treat cancer include the combination of cisplatin, doxorubicin, etoposide and cyclophosphamide, as well as the combination of cisplatin, doxorubicin, cyclophosphamide and vincristine.

The methods described above therefore find particular use in selecting appropriate treatment for early-stage cancer patients. The majority of individuals having cancer diagnosed at an early-stage of the disease enjoy long-term survival following surgery and/or radiation therapy without further adjuvant therapy. However, a significant percentage of these individuals will suffer disease recurrence or death, leading to clinical recommendations that some or all early-stage cancer patients should receive adjuvant therapy (e.g., chemotherapy). The methods of the present invention can identify this high-risk, poor prognosis population of individuals having early-stage cancer and thereby can be used to determine which ones would benefit from continued and/or more aggressive therapy and close monitoring following treatment. For example, individuals having early-stage cancer and assessed as having a poor prognosis by the methods disclosed herein may be selected for more aggressive adjuvant therapy, such as chemotherapy, following surgery and/or radiation treatment. In particular embodiments, the methods of the present invention may be used in conjunction with standard procedures and treatments to permit physicians to make more informed cancer treatment decisions.

The term “response to cancer therapy” or “outcome of cancer therapy” relates to any response of the hyperproliferative disorder (e.g., cancer) to a cancer therapy, preferably to a change in tumor mass and/or volume after initiation of neoadjuvant or adjuvant chemotherapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Response may also be assessed by caliper measurement or pathological examination of the tumor after biopsy or surgical resection for solid cancers. Responses may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more.

Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to copy number, level of expression, level of activity, etc. of one or more SNPs or indels described herein that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for whom the measurement values are known. In certain embodiments, the same doses of cancer therapeutic agents are administered to each subject. In related embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Outcomes can also be measured in terms of a “hazard ratio” (the ratio of death rates for one patient group to another; provides likelihood of death at a certain time point), “overall survival” (OS), and/or “progression free survival.” In certain embodiments, the prognosis comprises likelihood of overall survival rate at 1 year, 2 years, 3 years, 4 years, or any other suitable time point. The significance associated with the prognosis of poor outcome in all aspects of the present invention is measured by techniques known in the art. For example, significance may be measured with calculation of odds ratio. In a further embodiment, the significance is measured by a percentage. In one embodiment, a significant risk of poor outcome is measured as odds ratio of 0.8 or less or at least about 1.2, including by not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, and greater, or any range in between, with respect to a relevant outcome (e.g., accuracy, sensitivity, specificity, 5-year survival, 10-year survival, metastasis-free survival, stage prediction, and the like). In a further embodiment, a significant increase in risk is at least about 50%. Thus, the present invention further provides methods for making a treatment decision for a cancer patient, comprising carrying out the methods for prognosing a cancer patient according to the different aspects and embodiments of the present invention, and then weighing the results in light of other known clinical and pathological risk factors, in determining a course of treatment for the cancer patient. For example, a cancer patient that is shown by the methods of the invention to have an increased risk of poor outcome by combination chemotherapy treatment can be treated with more aggressive therapies, including but not limited to radiation therapy, peripheral blood stem cell transplant, bone marrow transplant, or novel or experimental therapies under clinical investigation. In addition, it will be understood that the cancer therapy responses can be predicted by the methods described herein according to enhanced sensitivity and/or specificity criteria. For example, sensitivity and/or specificity can be at least 0.80, 0.81, 0.2, 0.83, 0.84, 0.85, 0.86, 0.87, 0.88, 0.89, 0.90, 0.91, 0.92, 0.93, 0.94, 0.95, 0.96, 0.97, 0.98, 0.99 or greater, any range in between, or any combination for each of sensitivity and specificity.

The term “sensitize” means to alter cancer cells or tumor cells in a way that allows for more effective treatment of the associated cancer with a cancer therapy (e.g., chemotherapeutic or radiation therapy. In some embodiments, normal cells are not affected to an extent that causes the normal cells to be unduly injured by the cancer therapy (e.g., chemotherapy or radiation therapy). An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 25% or more, for example, 30%, 40%, 50%, 60%, 70%, 80%, or more, to 2-fold, 3-fold, 4-fold, 5-fold, 10-fold, 15-fold, 20-fold or more, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The present invention further provides novel therapeutic methods of preventing, delaying, reducing, and/or treating a cancer, including a cancerous tumor. In one embodiment, a method of treatment comprises administering to a subject (e.g., a subject in need thereof), an effective amount of a dual virus packaging system (i.e., rAAV (e.g., rAAV-Onco-CRISPR or rAAV-TSG) and Ad-rAAVpack) or the rAAV-Onco-CRISPR or rAAV-TSG, alone. A subject in need thereof may include, for example, a subject who has been diagnosed with a tumor, including a pre-cancerous tumor, a cancer, or a subject who has been treated, including subjects that have been refractory to the previous treatment.

The methods of the present invention may be used to treat any cancerous or pre-cancerous tumor. In certain embodiments, the cancerous tumor may be located in a tissue selected from brain, colon, urogenital, lung, renal, prostate, pancreas, liver, esophagus, stomach, hematopoietic, breast, thymus, testis, ovarian, skin, bone marrow and/or uterine tissue. In some embodiments, methods and compositions of the present invention may be used to treat any cancer. Cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The compositions described herein may be delivered by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The terms “systemic administration,” “administered systemically,” “peripheral administration,” and “administered peripherally” as used herein mean the administration of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, such that it enters the patient's system and, thus, is subject to metabolism and other like processes.

The terms “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection, intratumoral injection, and infusion.

In certain embodiments the pharmaceutical compositions are delivered generally (e.g., via oral or parenteral administration). In certain other embodiments the pharmaceutical compositions are delivered locally through direct injection into a tumor or direct injection into the tumor's blood supply (e.g., arterial or venous blood supply). In some embodiments, the pharmaceutical compositions are delivered by both a general and a local administration. For example, a subject with a tumor may be treated through direct injection of a composition containing a composition described herein into the tumor or the tumor's blood supply in combination with oral administration of a pharmaceutical composition of the present invention. If both local and general administration is used, local administration can occur before, concurrently with and/or after general administration.

In certain embodiments, the methods of treatment of the present invention, including treating a cancerous or pre-cancerous tumor comprise administering compositions described herein in combination with a second agent and/or therapy to the subject. By “in combination with” is meant the administration of the dual virus packaging system (i.e., rAAV (e.g., rAAV-Onco-CRISPR or rAAV-TSG) and Ad-rAAVpack)) or the rAAV-Onco-CRISPR or rAAV-TSG, alone, with one or more therapeutic agents either simultaneously, sequentially, or a combination thereof. Therefore, a subject administered a combination of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, and/or therapeutic agents, can receive the compositions comprising the dual virus packaging system as described herein, and one or more therapeutic agents at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 mins. or longer of one another. In other embodiments, agents administered sequentially, can be administered within 1, 5, 10, 15, 20 or more days of one another.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

Such methods in certain embodiments comprise administering pharmaceutical compositions comprising compositions described herein in conjunction with one or more chemotherapeutic agents and/or scavenger compounds, including chemotherapeutic agents described herein, as well as other agents known in the art. Conjunctive therapy includes sequential, simultaneous and separate, or co-administration of the composition in a way that the therapeutic effects of the compositions comprising the dual virus packaging system (i.e., rAAV (e.g., rAAV-Onco-CRISPR or rAAV-TSG) and Ad-rAAVpack)) administered have not entirely disappeared when the subsequent compound is administered. In one embodiment, the second agent is a chemotherapeutic agent. In another embodiment, the second agent is a scavenger compound. In another embodiment, the second agent is radiation therapy. In a further embodiment, radiation therapy may be administered in addition to the composition. In certain embodiments, the second agent may be co-formulated in the separate pharmaceutical composition.

In some embodiments, the subject pharmaceutical compositions of the present invention will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Dosage may be based on the amount of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, per kg body weight of the patient. For example, a range of amounts of compositions or compound encapsulated therein are contemplated, including about 0.001, 0.01, 0.1, 0.5, 1, 10, 15, 20, 25, 50, 75, 100, 150, 200 or 250 mg or more of such compositions per kg body weight of the patient. Other amounts will be known to those of skill in the art and readily determined.

In certain embodiments, the dosage of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, will generally be in the range of about 0.001 mg to about 250 mg per kg body weight, specifically in the range of about 50 mg to about 200 mg per kg, and more specifically in the range of about 100 mg to about 200 mg per kg. In one embodiment, the dosage is in the range of about 150 mg to about 250 mg per kg. In another embodiment, the dosage is about 200 mg per kg.

In some embodiments the molar concentration of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, in a pharmaceutical composition will be less than or equal to about 2.5 M, 2.4 M, 2.3 M, 2.2 M, 2.1 M, 2 M, 1.9 M, 1.8 M, 1.7 M, 1.6 M, 1.5 M, 1.4 M, 1.3 M, 1.2 M, 1.1 M, 1 M, 0.9 M, 0.8 M, 0.7 M, 0.6 M, 0.5 M, 0.4 M, 0.3 M or 0.2 M. In some embodiments the concentration of the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, will be less than or equal to about 0.10 mg/ml, 0.09 mg/ml, 0.08 mg/ml, 0.07 mg/ml, 0.06 mg/ml, 0.05 mg/ml, 0.04 mg/ml, 0.03 mg/ml or 0.02 mg/ml.

Actual dosage levels of the active ingredients in the compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular therapeutic agent in the formulation employed, or the ester, salt or amide thereof, the route of administration, the time of administration, the rate of excretion or metabolism of the particular therapeutic agent being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. All aspects of the treatment, including supplements, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments, for example, to the amount(s) of agent administered and to the time of administration may be made based on these reevaluations.

Treatment may be initiated with smaller dosages which are less than the optimum dose of the compound. Thereafter, the dosage may be increased by small increments until the optimum therapeutic effect is attained.

As described above, the composition comprising the dual virus packaging system (i.e., rAAV (e.g., rAAV-Onco-CRISPR or rAAV-TSG) and Ad-rAAVpack)) or the rAAV-Onco-CRISPR or rAAV-TSG, alone, may be administered in combination with radiation therapy. An optimized dose of radiation therapy may be given to a subject as a daily dose. Optimized daily doses of radiation therapy may be, for example, from about 0.25 to 0.5 Gy, about 0.5 to 1.0 Gy, about 1.0 to 1.5 Gy, about 1.5 to 2.0 Gy, about 2.0 to 2.5 Gy, and about 2.5 to 3.0 Gy. An exemplary daily dose may be, for example, from about 2.0 to 3.0 Gy. A higher dose of radiation may be administered, for example, if a tumor is resistant to lower doses of radiation. High doses of radiation may reach, for example, 4 Gy. Further, the total dose of radiation administered over the course of treatment may, for example, range from about 50 to 200 Gy. In an exemplary embodiment, the total dose of radiation administered over the course of treatment ranges, for example, from about 50 to 80 Gy. In certain embodiments, a dose of radiation may be given over a time interval of, for example, 1, 2, 3, 4, or 5 mins., wherein the amount of time is dependent on the dose rate of the radiation source.

In certain embodiments, a daily dose of optimized radiation may be administered, for example, 4 or 5 days a week, for approximately 4 to 8 weeks. In an alternate embodiment, a daily dose of optimized radiation may be administered daily seven days a week, for approximately 4 to 8 weeks. In certain embodiments, a daily dose of radiation may be given a single dose. Alternately, a daily dose of radiation may be given as a plurality of doses. In a further embodiment, the optimized dose of radiation may be a higher dose of radiation than can be tolerated by the patient on a daily base. As such, high doses of radiation may be administered to a patient, but in a less frequent dosing regimen.

The types of radiation that may be used in cancer treatment are well known in the art and include electron beams, high-energy photons from a linear accelerator or from radioactive sources such as cobalt or cesium, protons, and neutrons. An exemplary ionizing radiation is an x-ray radiation.

Methods of administering radiation are well known in the art. Exemplary methods include, but are not limited to, external beam radiation, internal beam radiation, and radiopharmaceuticals. In external beam radiation, a linear accelerator is used to deliver high-energy x-rays to the area of the body affected by cancer. Since the source of radiation originates outside of the body, external beam radiation can be used to treat large areas of the body with a uniform dose of radiation. Internal radiation therapy, also known as brachytherapy, involves delivery of a high dose of radiation to a specific site in the body. The two main types of internal radiation therapy include interstitial radiation, wherein a source of radiation is placed in the effected tissue, and intracavity radiation, wherein the source of radiation is placed in an internal body cavity a short distance from the affected area. Radioactive material may also be delivered to tumor cells by attachment to tumor-specific antibodies. The radioactive material used in internal radiation therapy is typically contained in a small capsule, pellet, wire, tube, or implant. In contrast, radiopharmaceuticals are unsealed sources of radiation that may be given orally, intravenously or directly into a body cavity.

Radiation therapy may also include stereotactic surgery or stereotactic radiation therapy, wherein a precise amount of radiation can be delivered to a small tumor area using a linear accelerator or gamma knife and three dimensional conformal radiation therapy (3DCRT), which is a computer assisted therapy to map the location of the tumor prior to radiation treatment.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the compositions comprising the dual virus packaging system (i.e., rAAV (e.g., rAAV-Onco-CRISPR or rAAV-TSG) and Ad-rAAVpack)) described herein relative to the rAAV-Onco-CRISPR or rAAV-TSG, alone. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the compositions comprising the dual virus packaging system (i.e., rAAV (e.g., rAAV-Onco-CRISPR or rAAV-TSG) and Ad-rAAVpack)) described herein relative to the rAAV-Onco-CRISPR or rAAV-TSG, alone. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the compositions comprising the dual virus packaging system (i.e., rAAV (e.g., rAAV-Onco-CRISPR or rAAV-TSG) and Ad-rAAVpack)) described herein relative to the rAAV-Onco-CRISPR or rAAV-TSG, alone. Although compounds that exhibit toxic side effects may be used, care should be taken to design a delivery system that targets the compounds to the desired site in order to reduce side effects.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% inhibition of cancer cell growth in an assay.

In any of the above-described methods, the administering of the compositions comprising the dual virus packaging system (i.e., rAAV (e.g., rAAV-Onco-CRISPR or rAAV-TSG) and Ad-rAAVpack)) can result in at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy in a subject, compared to the solid malignancy before administration of the compositions comprising the dual virus packaging system (i.e., rAAV (e.g., rAAV-Onco-CRISPR or rAAV-TSG) and Ad-rAAVpack)).

In some embodiments, the therapeutically effective amount of the compositions comprising the dual virus packaging system (i.e., rAAV (e.g., rAAV-Onco-CRISPR or rAAV-TSG) and Ad-rAAVpack)) is administered prophylactically to prevent a solid malignancy from forming in the subject.

In some embodiments, the subject is human. In other embodiments, the subject is non-human, such as a mammal.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ as determined in cell culture. Such information may be used to more accurately determine useful doses in humans. Levels in plasma may be measured, for example, by high performance liquid chromatography.

### IV. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments ±20%, in some embodiments+10%, in some embodiments ±5%, in some embodiments ±1%, in some embodiments+0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXEMPLIFICATIONS

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

A dual-virus packaging system for the in vivo replication of therapeutic adeno-associated viruses.

Background:

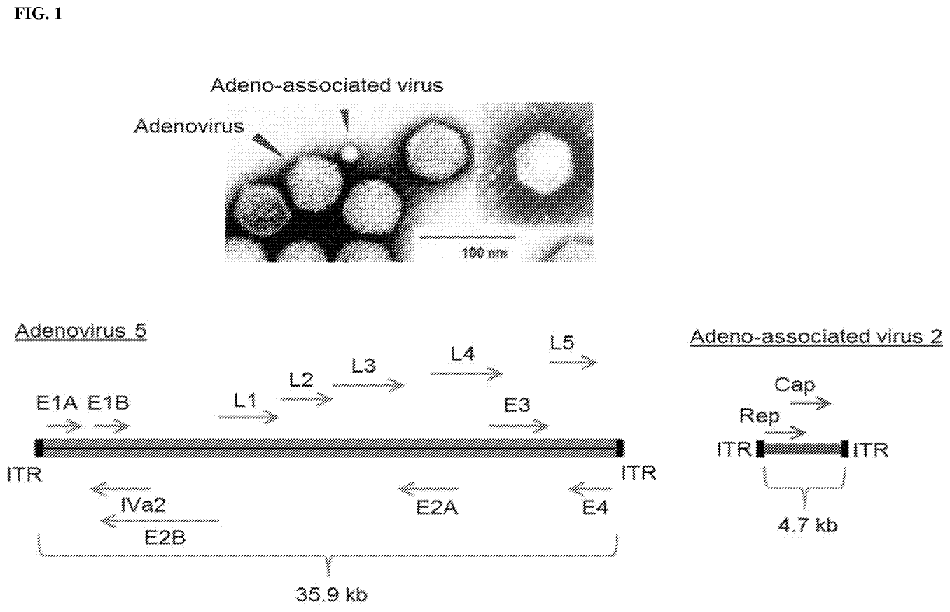

Recombinant adeno-associated viruses (rAAV) are the preferred vector for tissue-specific, in vivo gene therapy. These compact viruses are non-pathogenic and can infect both proliferative and quiescent cell populations with high efficiency. Wild-type AAV belong to the genus Dependoparvovirus, and were originally discovered in adenovirus (Ad)-infected cells. These simple viruses contain just two genes, rep and cap (FIG. 1). The remaining genes required for the AAV infectious cycle are provided in trans by Ad. In the design of therapeutic rAAV, the wild type viral genes are replaced by transgenes. rAAV must therefore be packaged in vitro. In the standard rAAV packaging system, several of the required trans-factors are provided by the packaging cell line 293, which was originally created by the transformation of human embryonic kidney cells with adenovirus DNA. The rest of the trans-factors—including the AAV rep and cap genes—are delivered on plasmids that are co-transfected along with the viral transgene construct. The only viral genetic elements retained in “gutless” rAAVs are the two inverted terminal repeats (ITRs). As a result, the infectious virus particles generated by in vitro packaging are replication-deficient.

Transgenes can be efficiently delivered by rAAV to tissues, but this is a “one-shot” process; no new virus is generated in the vicinity of the injection site. For many applications, a single administration of rAAV can modify a proportion of target cells that is sufficient to achieve a significant clinical response. For other applications, the proportion of cells that can be modified by a single rAAV treatment may be insufficient to achieve the desired response. This limitation is particularly relevant to the therapeutic use of rAAV against neoplastic disease, in which tissues are disordered and the number of target cells tends to increase.

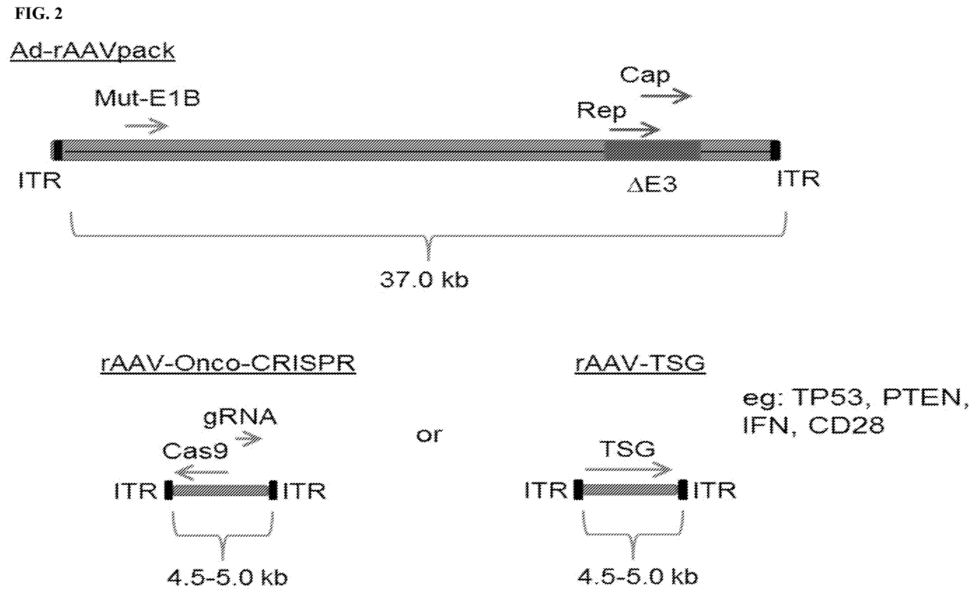

Provided herein is a viral system in which therapeutic rAAV can be iteratively replicated in vivo. At the core of this system is a novel derivative of Adenovirus 5 called Ad-rAAVpack, in which the rep and cap genes from wild type AAV replace the Ad E3 gene (FIG. 2). Ad E3 normally functions to allow the virus to evade host immune responses, but is not required for lytic infection nor for packaging of AAV. Because the rep-cap cassette is only ˜1 kb larger than the E3 gene, the total size of Ad-rAAVpack is well within the published Ad packaging capacity.

Ad-rAAVpack has all of the trans-elements required for the replication and packaging of a companion rAAV. Co-infection of target tissues with Ad-rAAVpack and a therapeutic rAAV would therefore permit the rAAV to be propagated in vivo, potentially increasing the efficiency of transgene delivery. Ultimately, the extent of the dual infection may be limited by the host immune response.

### Example 2

### Methods

Plasmid Construction:

To generate the H1 bidirectional construct, the human codon optimized Cas9 gene, and an SV40 terminator was fused to the 230 bp H1 promoter where the pol II transcript is endogenously found (minus strand). In between the H1 promoter and the gRNA scaffold, an AvrII site was engineered to allow for the insertion of targeting sequence. The SV40[rev]::hcas9[rev]::H::gRNA scaffold::pol III terminator sequence was then cloned into an Ndel/XbaI digest pUC19 vector. To generate the various gRNAs used in this study, overlapping oligos were annealed and amplified by PCR using two-step amplification Phusion Flash DNA polymerase (Thermo Fisher Scientific, Rockford, Ill.), and subsequently purified using Carboxylate-Modified Sera-Mag Magnetic Beads (Thermo Fisher Scientific) mixed with 2× volume 25% PEG and 1.5M NaCl. The purified PCR products were then resuspended in H₂O and quantitated using a NanoDrop 1000 (Thermo Fisher Scientific). The gRNA-expressing constructs were generated using the Gibson Assembly (New England Biolabs, Ipswich, Mass.) (Gibson et al. (2009) Nature Methods 6:343-345) with slight modifications. The total reaction volume was reduced from 20 μl to 2 μl.

Human embryonic kidney (HEK) cell line 293T (Life Technologies, Grand Island, N.Y.) was maintained at 37° C. with 5% CO₂/20% O₂ in Dulbecco's modified Eagle's Medium (DMEM) (Invitrogen) supplemented with 10% fetal bovine serum (Gibco, Life Technologies, Grand Island, N.Y.) and 2 mM GlutaMAX (Invitrogen).

Surveyor Assay and Sequencing Analysis for Genome Modification:

For Surveyor analysis, genomic DNA was extracted by resuspending cells in QuickExtract solution (Epicentre, Madison, Wis.), incubating at 65° C. for 15 min, and then at 98° C. for 10 min. The extract solution was cleaned using DNA Clean and Concentrator (Zymo Research, Irvine, Calif.) and quantitated by NanoDrop (Thermo Fisher Scientific). The genomic region surrounding the CRISPR target sites was amplified from 100 ng of genomic DNA using Phusion DNA polymerase (New England Biolabs). Multiple independent PCR reactions were pooled and purified using Qiagen MinElute Spin Column following the manufacturer's protocol (Qiagen, Valencia, Calif.). An 8 μl volume containing 400 ng of the PCR product in 12.5 mM Tris-HCl (pH 8.8), 62.5 mM KCl and 1.875 mM MgCl₂ was denatured and slowly reannealed to allow for the formation of heteroduplexes: 95° C. for 10 min, 95° C. to 85° C. ramped at −1.0° C./sec, 85° C. for 1 sec, 85° C. to 75° C. ramped at −1.0° C./sec, 75° C. for 1 sec, 75° C. to 65° C. ramped at −1.0° C./sec, 65° C. for 1 sec, 65° C. to 55° C. ramped at −1.0° C./sec, 55° C. for 1 sec, 55° C. to 45° C. ramped at −1.0° C./sec, 45° C. for 1 sec, 45° C. to 35° C. ramped at −1.0° C./sec, 35° C. for 1 sec, 35° C. to 25° C. ramped at −1.0° C./sec, and then held at 4° C. 1 μl of Surveyor Enhancer and 1 μl of Surveyor Nuclease (Transgenomic, Omaha, Nebr.) were added to each reaction, incubated at 42° C. for 60 min, after which, 1 μl of the Stop Solution was added to the reaction. 1 μl of the reaction was quantitated on the 2100 Bioanalyzer using the DNA 1000 chip (Agilent, Santa Clara, Calif.). For gel analysis, 2 μl of 6× loading buffer (New England Biolabs) was added to the remaining reaction and loaded onto a 3% agarose gel containing ethidium bromide. Gels were visualized on a Gel Logic 200 Imaging System (Kodak, Rochester, N.Y.), and quantitated using ImageJ v. 1.46. NHEJ frequencies were calculated using the binomial-derived equation:

$$\% gene modification = 1 - \sqrt{1 - \frac{( a + b )}{( a + b + c )}} \times 100 ;$$

where the values of “a” and “b” are equal to the integrated area of the cleaved fragments after background subtraction and “c” is equal to the integrated area of the un-cleaved PCR product after background subtraction (Guschin et al. (2010) Methods in Molecular Biology 649: 247-256).

A software was developed in-house (http://crispr.technology) to design unique gRNAs that anneal to recurrent oncogenic mutations. These gRNAs can direct the CRISPR/Cas9-mediated disruption of these mutant alleles. Intra-tumoral delivery of these gRNA along with a Cas9 protein may inhibit the growth of tumors that harbor these mutations. The specific oncogenes targeted in this manner are:

| Oncogene | Mutation | Gene-specific gRNA sequence |
|---|---|---|
| KRAS | G13D | GTAGTTGGAGCTGGTGACGTAGG |
| | | (SEQ ID NO: 1) |
| KRAS | G12C | GTAGTTGGAGCTTGTGGCGTAGG |
| | | (SEQ ID NO: 2) |
| KRAS | G12D | GTAGTTGGAGCTGATGGCGTAGG |
| | | (SEQ ID NO: 3) |
| PIK3CA | E345K | TCTCTCTGAAATCACTAAGCAGG |
| | | (SEQ ID NO: 4) |
| PIK3CA | D549N | AAGATTTTCTATGGAGTCACAGG |
| | | (SEQ ID NO: 5) |
| PIK3CA | H1047R | CAAATGAATGATGCACGTCATGG |
| | | (SEQ ID NO: 6) |
| IDH1 | R132H | ATCATAGGTCGTCATGCTTATGG |
| | | (SEQ ID NO: 7) |
| | R132H | TCATAGGTCGTCATGCTTATGGG |
| | | (SEQ ID NO: 8) |
| | R132H | CATAGGTCGTCATGCTTATGGGG |
| | | (SEQ ID NO: 9) |
| | R132H | GCATGACGACCTATGATGATAGG |
| | | (SEQ ID NO: 10) |

Collectively, these specific mutations in KRAS and PIK3CA are found in the majority of cancers in the lung and throughout the GI tract. The IDH1 R132 mutation is found in about 30% of gliomas. These brain tumors are particularly refractory to all conventional forms of therapy. Each of these gRNA primarily target the mutant allele.

HumanH1::target:gRNA scaffold
Target: WT KRAS
(SEQ ID NO: 11)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCGTAGTTGGAGCTGGT
GGCGTGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: KRAS G12C
(SEQ ID NO: 12)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCGTAGTTGGAGCTTGT
GGCGTGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: KRAS G12D
(SEQ ID NO: 13)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCGTAGTTGGAGCTGAT
GGCGTGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: KRAS G13D
(SEQ ID NO: 14)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCGTAGTTGGAGCTGGT
GACGTGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: WT PIK3CA
(SEQ ID NO: 15)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCTCTCTCTGAAATCAC
TGAGCGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: PIK3CA E545K
(SEQ ID NO: 16)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCTCTCTCTGAAATCAC
TAAGCGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: PIK3CA E549N
(SEQ ID NO: 17)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCAAGATTTTCTATGGA
GTCACGTTTTAGAGCTAGAAATAGCAAGTTAAAATAAGGCTAGTCCGTT
ATCAACTTGAAAAAGTGGCACCGAGTCGGTGC
HumanH1::target:gRNA scaffold
Target: PIK3CA H1047R
(SEQ ID NO: 18)
GGAATTCGAACGCTGACGTCATCAACCCGCTCCAAGGAATCGCGGGCCC
AGTGTCACTAGGCGGGAACACCCAGCGCGCGTGCGCCCTGGCAGGAAGA
TGGCTGTGAGGGACAGGGGAGTGGCGCCCTGCAATATTTGCATGTCGCT
ATGTGTTCTGGGAAATCACCATAAACGTGAAATGTCTTTGGATTTGGGA
ATCTTATAAGTTCTGTATGAGACCACTTTTTCCCCAAATGAATGATGCA
CGTCAGTTTTAGAGCTAGAAATAGCAAGTTAAAATAAGGCTAGTCCGTT
ATCAACTTGAAAAAGTGGCACCGAGTCGGTGC

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Although the foregoing subject matter has been described in some detail by way of illustration and example for purposes of clarity of understanding, it will be understood by those skilled in the art that certain changes and modifications can be practiced within the scope of the appended claims.

1. A method for preventing, inhibiting, or treating cancer in a subject in need thereof, the method comprising:
(a) providing a non-naturally occurring nuclease system comprising one or more vectors comprising:
i) a promoter operably linked to at least one nucleotide sequence encoding a nuclease system guide RNA (gRNA), wherein the gRNA hybridizes with a target sequence of a DNA molecule in a cell of the subject, and wherein the DNA molecule encodes one or more oncogene products expressed in the cell; and
ii) a regulatory element operable in a cell operably linked to a nucleotide sequence encoding a genome-targeted nuclease,
wherein components (i) and (ii) are located on the same or different vectors of the system, wherein the gRNA targets and hybridizes with the target sequence and the nuclease cleaves one or both strands of the DNA molecule to alter expression of the one or more gene products; and
(b) administering to the subject a therapeutically effective amount of the system.

2. The method of claim 1, further comprising the step of providing a recombinant adeno-associated virus-packaging adenovirus (Ad-rAAVpack).

3. The method of claim 2, wherein the Ad-rAAVpack is provided concurrently or co-administered with the nuclease system.

4. The method of claim 1, wherein the system is CRISPR-Cas9.

5. The method of claim 1, wherein the system is packaged into a single adeno-associated virus (AAV) particle.

6. The method of claim 1, wherein the adeno-associated virus-packaging adenovirus comprises at least one deletion in an adenoviral gene.

7. The method of claim 6, wherein the adeno-associated virus-packaging adenovirus is selected from adenovirus serotype 2, adenovirus serotype 5, or adenovirus serotype 35.

8. (canceled)

9. The method of claim 6, wherein the adenoviral gene is selected from E1A, E1B, E2A, E2B, E3, E4, L1, L2, L3, L4, or L5.

10. (canceled)

11. The method of claim 1, wherein the system inactivates one or more gene products.

12. The method of claim 1, wherein the nuclease system excises at least one gene mutation.

13. The method of claim 1, wherein the promoter is a H1 promoter.

14. The method of claim 13, wherein the H1 promoter is bidirectional.

15. The method of claim 14, wherein the H1 promoter comprises:
a) control elements that provide for transcription in one direction of the at least one nucleotide sequence encoding the gRNA; and
b) control elements that provide for transcription in the opposite direction of the nucleotide sequence encoding the genome-targeted nuclease.

16. The method of claim 1, wherein the genome-targeted nuclease is Cas9 protein.

17. The method of claim 16, wherein the Cas9 protein is codon optimized for expression in the cell.

18. The method of claim 13, wherein the promoter is operably linked to at least one, two, three, four, five, six, seven, eight, nine, or ten gRNA.

19. The method of claim 1, wherein the target sequence is an oncogene or tumor suppressor gene.

20. The method of claim 1, wherein the target sequence is an oncogene comprising at least one mutation.

21. (canceled)

22. The method of claim 20, wherein the target sequence is an oncogene selected from KRAS, PIK3CA, or IDH1.

23. The method of claim 22, wherein the target sequence is an oncogene, said oncogene is KRAS.

24. The method of claim 23, wherein the KRAS comprises a mutation selected from G13D, G12C, or G12D.

25. The method of claim 23, wherein the target sequence is selected from the group consisting of SEQ ID NO: 12-14, or combinations thereof.

26. The method of claim 22, wherein the target sequence is an oncogene, said oncogene is PIK3CA.

27. The method of claim 26, wherein the PIK3CA comprises a mutation selected from E345K, D549N, or H1047R.

28. The method of claim 26, wherein the target sequence is selected from the group consisting of SEQ ID NO: 16-18, or combinations thereof.

29. The method of claim 22, wherein the target sequence is an oncogene, said oncogene IDH1.

30. The method of claim 29, wherein the IDH1 comprises a R132H mutation.

31. The method of claim 1, wherein the gRNA sequence is selected from the group consisting of the nucleotide sequences set forth in SEQ ID NO: 1-10, or combinations thereof.

32.-46. (canceled)

47. A method of altering expression of one or more gene products in a cell, wherein the cell comprises a DNA molecule encoding the one or more gene products, the method comprising introducing into the cell:
(i) a non-naturally occurring nuclease system comprising one or more vectors comprising:
a) a promoter operably linked to at least one nucleotide sequence encoding a nuclease system guide RNA (gRNA), wherein the gRNA hybridizes with a target sequence of the DNA molecule; and
b) a regulatory element operable in the cell operably linked to a nucleotide sequence encoding a genome-targeted nuclease,
wherein components (a) and (b) are located on the same or different vectors of the system, wherein the gRNA targets and hybridizes with the target sequence and the nuclease cleaves one or both strands of the DNA molecule to alter expression of the one or more gene products.

48.-83. (canceled)


SEQUENCE LISTING (20 sequences)

SEQ ID NO: 1 - DNA, 23 bp, Artificial Sequence
  Description of Artificial Sequence Synthetic oligonucleotide
gtagttggagctggtgacgtagg

SEQ ID NO: 2 - DNA, 23 bp, Artificial Sequence
  Description of Artificial Sequence Synthetic oligonucleotide
gtagttggagcttgtggcgtagg

SEQ ID NO: 3 - DNA, 23 bp, Artificial Sequence
  Description of Artificial Sequence Synthetic oligonucleotide
gtagttggagctgatggcgtagg

SEQ ID NO: 4 - DNA, 23 bp, Artificial Sequence
  Description of Artificial Sequence Synthetic oligonucleotide
tctctctgaaatcactaagcagg

SEQ ID NO: 5 - DNA, 23 bp, Artificial Sequence
  Description of Artificial Sequence Synthetic oligonucleotide
aagattttctatggagtcacagg

SEQ ID NO: 6 - DNA, 23 bp, Artificial Sequence
  Description of Artificial Sequence Synthetic oligonucleotide
caaatgaatgatgcacgtcatgg

SEQ ID NO: 7 - DNA, 23 bp, Artificial Sequence
  Description of Artificial Sequence Synthetic oligonucleotide
atcataggtcgtcatgcttatgg

SEQ ID NO: 8 - DNA, 23 bp, Artificial Sequence
  Description of Artificial Sequence Synthetic oligonucleotide
tcataggtcgtcatgcttatggg

SEQ ID NO: 9 - DNA, 23 bp, Artificial Sequence
  Description of Artificial Sequence Synthetic oligonucleotide
cataggtcgtcatgcttatgggg

SEQ ID NO: 10 - DNA, 23 bp, Artificial Sequence
  Description of Artificial Sequence Synthetic oligonucleotide
gcatgacgacctatgatgatagg

SEQ ID NO: 11 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccgtagttggagctggtggcgtgttttagagctagaaatagcaagttaaaataaggctagtccgttatcaacttgaaaaagtggcaccgagtcggtgc

SEQ ID NO: 12 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccgtagttggagcttgtggcgtgttttagagctagaaatagcaagttaaaataaggctagtccgttatcaacttgaaaaagtggcaccgagtcggtgc

SEQ ID NO: 13 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccgtagttggagctgatggcgtgttttagagctagaaatagcaagttaaaataaggctagtccgttatcaacttgaaaaagtggcaccgagtcggtgc

SEQ ID NO: 14 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccgtagttggagctggtgacgtgttttagagctagaaatagcaagttaaaataaggctagtccgttatcaacttgaaaaagtggcaccgagtcggtgc

SEQ ID NO: 15 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttccctctctctgaaatcactgagcgttttagagctagaaatagcaagttaaaataaggctagtccgttatcaacttgaaaaagtggcaccgagtcggtgc

SEQ ID NO: 16 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttccctctctctgaaatcactaagcgttttagagctagaaatagcaagttaaaataaggctagtccgttatcaacttgaaaaagtggcaccgagtcggtgc

SEQ ID NO: 17 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttcccaagattttctatggagtcacgttttagagctagaaatagcaagttaaaataaggctagtccgttatcaacttgaaaaagtggcaccgagtcggtgc

SEQ ID NO: 18 - DNA, 326 bp, Artificial Sequence
  Description of Artificial Sequence Synthetic polynucleotide
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactttttccccaaatgaatgatgcacgtcagttttagagctagaaatagcaagttaaaataaggctagtccgttatcaacttgaaaaagtggcaccgagtcggtgc

SEQ ID NO: 19 - DNA, 122 bp, Artificial Sequence
  Description of Artificial Sequence Synthetic polynucleotide
  CDS (12)..(122)
gcctgctgaaaatgactgaatataaacttgtggtagttggagctggtgacgtaggcaagagtgccttgacgatacagctaattcagaatcattttgtggacgaatatgatccaacaatagag

SEQ ID NO: 20 - protein, 37 aa, Artificial Sequence
  Description of Artificial Sequence Synthetic polypeptide
MTEYKLVVVGAGDVGKSALTIQLIQNHFVDEYDPTIE